(19) United States
(12) Patent Application Publication
(10) Pub. No.: US 2016/0272967 A1
(43) Pub. Date: Sep. 22, 2016

(54) NUCLEIC ACID, PHARMACEUTICAL COMPOSITION AND USES THEREOF

(71) Applicant: SUZHOU RIBO LIFE SCIENCE CO., LTD., Kunshan (CN)

(72) Inventors: Ge ZHANG, Kowloon (HK); Aiping LV, Kowloon (HK); Baosheng GUO, Kowloon (HK); Hongyan ZHANG, Kunshan (CN)

(73) Assignee: SUZHOU RIBO LIFE SCIENCE CO., LTD., Kunshan (CN)

(21) Appl. No.: 14/914,830

(22) PCT Filed: Aug. 26, 2014
(86) PCT No.: PCT/CN2014/085170

§ 371 Date: Feb. 26, 2016

(30) Foreign Application Priority Data
Aug. 26, 2013 (CN) 201310375603.5

(51) Int. Cl. C12N 15/113; A61K 9/127

(52) U.S. Cl. CPC C12N 15/113; A61K 9/127; C12N 2310/14; C12N 2310/321

(57) ABSTRACT

Provided are a small interfering nucleic acid against bone formation inhibiting gene CKIP-1, a pharmaceutical composition thereof, and uses thereof in preparation of a pharmaceutical composition for treating and/or preventing diseases related to the abnormal expression of CKIP-1 gene. The small interfering nucleic acid is capable of cross-species inhibiting the CKIP-1 gene expression, inhibiting CKIP-1 expression in human, rhesus, rats and mice simultaneously, and facilitating the differentiation of osteoblasts and mineralization of bone matrix effectively.

### FIELD OF THE INVENTION

The invention relates to the field of biotechnology and specifically relates to a nucleic acid, uses of the nucleic acid and a pharmaceutical composition.

### BACKGROUND OF THE INVENTION

Osteoporosis is a systematic bone disease which is characterized by decrease in bone mass and damages to trabecular bone, represented by increase in brittleness of the bone, thereby being very liable to fracture. After the bone of an adult develops to maturity, metabolism of the bone is maintained through two interrelated processes, namely bone resorption and bone formation. With the aging of a human body, the ability of bone formation goes down, thereby being incapable of making up for bone resorption, resulting in bone loss and causing osteoporosis and fracture complications. With the increasingly serious aging of population, the incidence of osteoporosis is in an ascending trend year by year, thereby posing a serious threat to the health of patients.

In addition to basic drugs for preventing and treating osteoporosis, such as calcium agents and vitamin D, there are also bone turnover inhibitors, bone formation promoters, uncoupling agents and other therapeutic drugs. Most of these therapeutic drugs can maintain bone mass by inhibiting bone resorption, but only a small quantity of the drugs can promote the formation of new bone; and after long-term use, it may increase the risk of bone resorption or have other side effects. Thus, in the art, it needs to develop new therapeutic drugs that can promote the generation of new bone without stimulating bone resorption to achieve the effect of treating and even reversing the process of osteoporosis and simultaneously reduce the side effects caused by medication.

RNA interference (RNAi) is the natural process of post-transcriptional specific gene silencing mediated by double-stranded RNA. Theoretically, any gene-related disease can be treated with RNAi, and osteoporosis is no exception. Researches indicate that casein kinase-2 interaction protein 1 (CKIP-1) is a bone formation inhibiting gene. Importantly, CKIP-1 can specifically regulate bone formation rather than bone resorption, and its expression level in the bone samples of patients with rheumatoid arthritis late-stage bone destruction and patients with osteoporosis is higher than that in the bone samples of normal people. It can be inferred that the small nucleic acid drugs targeting CKIP-1 will be a good variety for treating osteoporosis.

According to drug registration guidelines (USA) of Food and Drug Administration (FDA), the anti-osteoporosis drugs should be pre-clinically tested in two different animal models before clinical trial begin, e.g. at least covering rodents (rats or mice) and non-human primates (rhesus). However, the specific CKIP-1 siRNA targeting a certain species represents lower mRNA inhibition efficiency in other species, obviously, this is particularly not conductive to screening and researching of the drugs. Thus, it needs to find the CKIP-1-targeting siRNA which can represent relatively high mRNA inhibition efficiency in different species.

### SUMMARY OF THE INVENTION

The objectives of the invention are to overcome the shortcoming that existing nucleic acid hardly represents relatively high inhibition efficiency in different species, and provide a cross-species homologous small interfering nucleic acid which can target CKIP-1 gene and further achieve the effect of treating and/or preventing osteoporosis.

In order to achieve the object, the invention provides a nucleic acid, which contains at least one of siRNA-1 with a sense strand sequence which is a sequence having sequence identity of more than 90% with SEQ ID NO: 1 and an antisense strand sequence which is the sequence having the sequence identity of more than 90% with SEQ ID NO: 2, siRNA-2 with the sense strand sequence which is the sequence having the sequence identity of more than 90% with SEQ ID NO: 3 and the antisense strand sequence which is the sequence having the sequence identity of more than 90% with SEQ ID NO: 4, siRNA-3 with the sense strand sequence which is the sequence having the sequence identity of more than 90% with SEQ ID NO: 5 and the antisense strand sequence which is the sequence having the sequence identity of more than 90% with SEQ ID NO: 6, siRNA-4 with the sense strand sequence which is the sequence having the sequence identity of more than 90% with SEQ ID NO: 7 and the antisense strand sequence which is the sequence having the sequence identity of more than 90% with SEQ ID NO: 8, siRNA-5 with the sense strand sequence which is the sequence having the sequence identity of more than 90% with SEQ ID NO: 9 and the antisense strand sequence which is the sequence having the sequence identity of more than 90% with SEQ ID NO: 10, siRNA-6 with the sense strand sequence which is the sequence having the sequence identity of more than 90% with SEQ ID NO: 11 and the antisense strand sequence which is the sequence having the sequence identity of more than 90% with SEQ ID NO: 12, siRNA-7 with the sense strand sequence which is the sequence having the sequence identity of more than 90% with SEQ ID NO: 13 and the antisense strand sequence which is the sequence having the sequence identity of more than 90% with SEQ ID NO: 14 and siRNA-8 with the sense strand sequence which is the sequence having the sequence identity of more than 90% with SEQ ID NO: 15 and the antisense strand sequence which is the sequence having the sequence identity of more than 90% with SEQ ID NO: 16. Preferably, the sequence having the sequence identity of more than 90% refers to the sequence which is totally consistent or has only one base inconsistency, and more preferably, in the sense strand, one inconsistent base is positioned at position 19 of the sense strand, and in the antisense strand, one inconsistent base is positioned at position 1 of the antisense strand. All nucleotide groups in the small interfering nucleic acid can be the nucleotide groups without chemical modification and can also be the nucleotide groups containing at least one modification.

The invention further provides another nucleic acid, which is a plasmid inserted with a nucleic acid fragment encoding short hairpin ribonucleic acid. The plasmid expresses the short hairpin ribonucleic acid, and the nucleic acid fragment encoding the short hairpin ribonucleic acid comprises two short inverted repeat fragments and a loop fragment positioned between the two short inverted repeat fragments; the sequences of the two short inverted repeat fragments are the sequence having the sequence identity of more than 90% with SEQ ID NO: 17 and the sequence having the sequence identity of more than 90% with SEQ ID NO: 18 respectively, or the sequences of the two short inverted repeat fragments are the sequence having the sequence identity of more than 90% with SEQ ID NO: 19 and the sequence having the sequence identity of more than 90% with SEQ ID NO: 20 respectively, or the sequences of the short inverted repeat fragments are the sequence having the sequence identity of more than 90% with SEQ ID NO: 21 and the sequence having the sequence identity of more than 90% with SEQ ID NO: 22 respectively, or the sequences of the two short inverted repeat fragments are the sequence having the sequence identity of more than 90% with SEQ ID NO: 23 and the sequence having the sequence identity of more than 90% with SEQ ID NO: 24 respectively, or the sequences of the two short inverted repeat fragments are the sequence having the sequence identity of more than 90% with SEQ ID NO: 25 and the sequence having the sequence identity of more than 90% with SEQ ID NO: 26 respectively, or the sequences of the two short inverted repeat fragments are the sequence having the sequence identity of more than 90% with SEQ ID NO: 27 and the sequence having the sequence identity of more than 90% with SEQ ID NO: 28 respectively, or the sequences of the two short inverted repeat fragments are the sequence having the sequence identity of more than 90% with SEQ ID NO: 29 and the sequence having the sequence identity of more than 90% with SEQ ID NO: 30 respectively, or the sequences of the two short inverted repeat fragments are the sequence having the sequence identity of more than 90% with SEQ ID NO: 31 and the sequence having the sequence identity of more than 90% with SEQ ID NO: 32 respectively. Preferably, the sequence having the sequence identity of more than 90% refers to the sequence which is totally consistent or has only one base inconsistency, and more preferably, in the sense strand, one inconsistent base is positioned at position 19 of the sense strand, and in the antisense strand, one inconsistent base is positioned at position 1 of the antisense strand.

The invention further provides a separated target sequence of small interfering nucleic acid molecules of the CKIP-1 gene, wherein the sequence of the target sequence is the sequence having sequence identity of more than 90% with any one in SEQ ID NO: 33-40. Preferably, the sequence having the sequence identity of more than 90% refers to the sequence which is totally consistent or has only one base inconsistency, and more preferably, one inconsistent base is positioned at position 19 of the target sequence.

The invention further provides a pharmaceutical composition, which contains the nucleic acid as described above and a pharmaceutically acceptable carrier.

The invention further provides use of the nucleic acid in preparation of a pharmaceutical composition for treating and/or preventing diseases related to the abnormal expression of CKIP-1 gene.

The invention further provides a method for treating and/or preventing diseases related to the abnormal expression of CKIP-1 gene, the method comprising performing administration on a patient by using the nucleic acid and/or the pharmaceutical composition.

In addition, the invention further provides a method for inhibiting the expression of CKIP-1 gene in cells, the method comprising introducing the nucleic acid and/or the pharmaceutical composition into the cells.

Through the technical proposal, the nucleic acid and the pharmaceutical composition provided by the invention represent relatively high CKIP-1 inhibition efficiency in human, rhesus, rats and mice, and can effectively promote osteoblast differentiation and bone matrix mineralization and have a therapeutic and/or prophylactic effect on the diseases related to the abnormal expression of CKIP-1 gene.

Other features and advantages of the invention will be described in detail in the following detailed description of the embodiments.

### DETAILED DESCRIPTION OF THE EMBODIMENTS

The specific embodiments of the invention will be described below in detail. It should be understood that the specific embodiments described herein are only intended to illustrate and explain the invention instead of limiting the invention.

In the invention, unless otherwise indicated, the term siRNA used in the invention refers to small interfering ribonucleic acid, and shRNA refers to short hairpin ribonucleic acid.

A nucleic acid provided by the invention contains at least one of siRNA-1 with a sense strand sequence which is a sequence having sequence identity of more than 90% with SEQ ID NO: 1 and an antisense strand sequence which is the sequence having the sequence identity of more than 90% with SEQ ID NO: 2, siRNA-2 with the sense strand sequence which is the sequence having the sequence identity of more than 90% with SEQ ID NO: 3 and the antisense strand sequence which is the sequence having the sequence identity of more than 90% with SEQ ID NO: 4, siRNA-3 with the sense strand sequence which is the sequence having the sequence identity of more than 90% with SEQ ID NO: 5 and the antisense strand sequence which is the sequence having the sequence identity of more than 90% with SEQ ID NO: 6, siRNA-4 with the sense strand sequence which is the sequence having the sequence identity of more than 90% with SEQ ID NO: 7 and the antisense strand sequence which is the sequence having the sequence identity of more than 90% with SEQ ID NO: 8, siRNA-5 with the sense strand sequence which is the sequence having the sequence identity of more than 90% with SEQ ID NO: 9 and the antisense strand sequence which is the sequence having the sequence identity of more than 90% with SEQ ID NO: 10, siRNA-6 with the sense strand sequence which is the sequence having the sequence identity of more than 90% with SEQ ID NO: 11 and the antisense strand sequence which is the sequence having the sequence identity of more than 90% with SEQ ID NO: 12, siRNA-7 with the sense strand sequence which is the sequence having the sequence identity of more than 90% with SEQ ID NO: 13 and the antisense strand sequence which is the sequence having the sequence identity of more than 90% with SEQ ID NO: 14 and siRNA-8 with the sense strand sequence which is the sequence having the sequence identity of more than 90% with SEQ ID NO: 15 and the antisense strand sequence which is the sequence having the sequence identity of more than 90% with SEQ ID NO: 16.

Preferably, the sequence identity of more than 90% means that one base inconsistency exists between the sequences, in the sense strand, one inconsistent base is positioned at position 19 of the sense strand, and in the antisense strand, one inconsistent base is positioned at position 1 of the antisense strand.

More preferably, the nucleic acid of the invention contains at least one of siRNA-1 with the sense strand sequence of SEQ ID NO: 1 and the antisense strand sequence of SEQ ID NO: 2, siRNA-2 with the sense strand sequence of SEQ ID NO: 3 and the antisense strand sequence of SEQ ID NO: 4, siRNA-3 with the sense strand sequence of SEQ ID NO: 5 and the antisense strand sequence of SEQ ID NO: 6, siRNA-4 with the sense strand sequence of SEQ ID NO: 7 and the antisense strand sequence of SEQ ID NO: 8, siRNA-5 with the sense strand sequence of SEQ ID NO: 9 and the antisense strand sequence of SEQ ID NO: 10, siRNA-6 with the sense strand sequence of SEQ ID NO: 11 and the antisense strand sequence of SEQ ID NO: 12, siRNA-7 with the sense strand sequence of SEQ ID NO: 13 and the antisense strand sequence of SEQ ID NO: 14, siRNA-8 with the sense strand sequence of SEQ ID NO: 15 and the antisense strand sequence of SEQ ID NO: 16, siRNA-1A with the sense strand sequence of SEQ ID NO: 83 and the antisense strand sequence of SEQ ID NO: 84, siRNA-1G with the sense strand sequence of SEQ ID NO: 85 and the antisense strand sequence of SEQ ID NO: 86, siRNA-1C with the sense strand sequence of SEQ ID NO: 87 and the antisense strand sequence of SEQ ID NO: 88, siRNA-3A with the sense strand sequence of SEQ ID NO: 89 and the antisense strand sequence of SEQ ID NO: 90, siRNA-3U with the sense strand sequence of SEQ ID NO: 91 and the antisense strand sequence of SEQ ID NO: 92, siRNA-3C with the sense strand sequence of SEQ ID NO: 93 and the antisense strand sequence of SEQ ID NO: 94, siRNA-5A with the sense strand sequence of SEQ ID NO: 95 and the antisense strand sequence of SEQ ID NO: 96, siRNA-5U with the sense strand sequence of SEQ ID NO: 97 and the antisense strand sequence of SEQ ID NO: 98 and siRNA-5C with the sense strand sequence of SEQ ID NO: 99 and the antisense strand sequence of SEQ ID NO: 100. In this case, the sense strands and the antisense strands of siRNA-1A, siRNA-1G and siRNA-1C have sequence identity of 90% with the sense strand and the antisense strand of siRNA-1, respectively; the sense strands and the antisense strands of siRNA-3A, siRNA-3U and siRNA-3C have sequence identity of 90% with the sense strand and the antisense strand of siRNA-3, respectively; and the sense strands and the antisense strands of siRNA-5A, siRNA-5U and siRNA-5C have sequence identity of 90% with the sense strand and the antisense strand of siRNA-5, respectively.

According to the nucleic acid of the invention, wherein a nucleotide group contained in the nucleic acid is used as a basic structural unit. The nucleotide group contains a phosphoric acid group, a ribose group and a base, and preferably, the nucleic acid contains at least one modified nucleotide group. The modified nucleotide group can not cause the loss of function that the nucleic acid inhibits the expression of CKIP-1.

According to the nucleic acid of the invention, wherein the modified nucleotide group is the nucleotide group with a modified phosphoric acid group and/or a ribose group.

For example, the modification of the phosphoric acid group refers to modification of oxygen in the phosphoric acid group, including phosphorthioate modification and boranophosphate modification. As shown in the following formulas, the oxygen in the phosphoric acid group is replaced by sulfur and borane respectively. Both the two modifications can stabilize the structure of the nucleic acid, keeping high specificity and high affinity of base pairing.

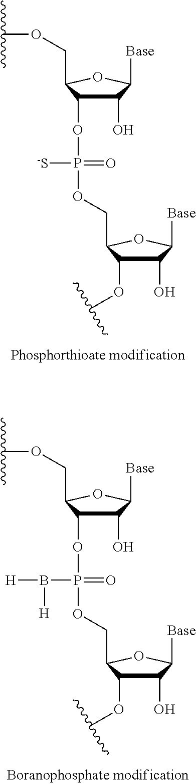

The modification of the ribose group refers to the modification of 2′-hydroxy group (2′-OH) in the ribose group. After introduction of a certain substituent, such as methoxy or fluoro group at the position of 2′-hydroxy group in the ribose group, ribonuclease in serum is less liable to cutting the nucleic acid so as to increase the stability of the nucleic acid and enable the nucleic acid to have stronger performance of resisting hydrolysis of nuclease. The modification of 2′-hydroxy group in nucleotide pentose comprises 2′-fluro modification, 2′-OME modification, 2′-MOE modification, 2′-DNP modification, LNA modification, 2′-Amino modification, 2′-Deoxy modification and the like.

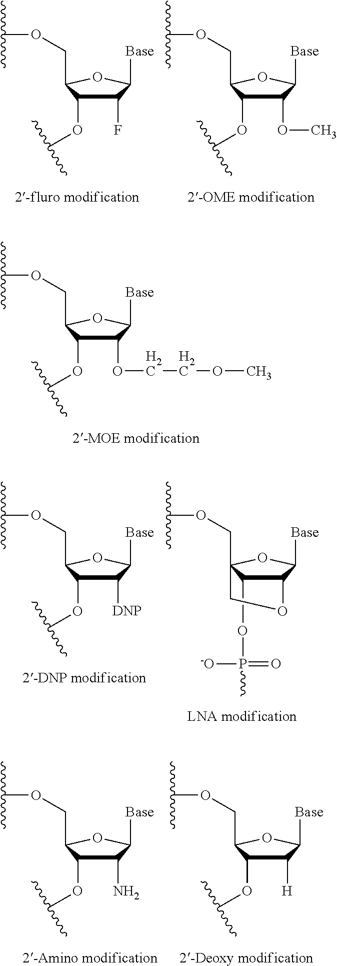

According to the nucleic acid of the invention, wherein preferably, the nucleotide group with the modified ribose group is the nucleotide group with the ribose group of which 2′-OH is substituted by the methoxy or fluoro group.

According to a particularly preferred embodiment of the invention, wherein the nucleotide group containing a uracil base or a cytosine base in the sense strand of the nucleic acid is the nucleotide group with the modified ribose group, namely the 2′-OH of the ribose group in the nucleotide group containing the uracil base or the cytosine base in the sense strand of the nucleic acid is substituted by the methoxy or fluoro group. More preferably, 3′ ends of the sense strand and the antisense strand of the nucleic acid are connected with dTdT, respectively. The nucleic acid with the above modification represents more excellent in-vivo inhibition effect, and the above modifications can further reduce in-vivo immunogenicity of the nucleic acid of the invention. For the specific modifications, reference can be made to Table 2.

According to the nucleic acid of the invention, wherein the siRNA including the siRNA-1, the siRNA-2, the siRNA-3, the siRNA-4, the siRNA-5, the siRNA-6, the siRNA-7 and the siRNA-8 can be obtained through a conventional method in the art, for example, it can be obtained by solid-phase synthesis or liquid-phase synthesis, and the solid-phase synthesis has commercial service already and are thus commercially available (such as Suzhou Ribo Life Science Co., Ltd.). The modified nucleotide group can be introduced through a nucleotide monomer with the corresponding modification.

Based on the above synthesized siRNA, the invention further provides an shRNA expression plasmid with the same or similar function with the above siRNA.

The invention further provides a nucleic acid, which is a plasmid inserted with a nucleic acid fragment encoding short hairpin ribonucleic acid. The plasmid expresses the short hairpin ribonucleic acid, and the nucleic acid fragment encoding the short hairpin ribonucleic acid comprises two short inverted repeat fragments and a loop fragment positioned between the two short inverted repeat fragments; the sequences of the two short inverted repeat fragments are the sequence having the sequence identity of more than 90% with SEQ ID NO: 17 and the sequence having the sequence identity of more than 90% with SEQ ID NO: 18 respectively, or the sequences of the two short inverted repeat fragments are the sequence having the sequence identity of more than 90% with SEQ ID NO: 19 and the sequence having the sequence identity of more than 90% with SEQ ID NO: 20 respectively, or the sequences of the short inverted repeat fragments are the sequence having the sequence identity of more than 90% with SEQ ID NO: 21 and the sequence having the sequence identity of more than 90% with SEQ ID NO: 22 respectively, or the sequences of the two short inverted repeat fragments are the sequence having the sequence identity of more than 90% with SEQ ID NO: 23 and the sequence having the sequence identity of more than 90% with SEQ ID NO: 24 respectively, or the sequences of the two short inverted repeat fragments are the sequence having the sequence identity of more than 90% with SEQ ID NO: 25 and the sequence having the sequence identity of more than 90% with SEQ ID NO: 26 respectively, or the sequences of the two short inverted repeat fragments are the sequence having the sequence identity of more than 90% with SEQ ID NO: 27 and the sequence having the sequence identity of more than 90% with SEQ ID NO: 28 respectively, or the sequences of the two short inverted repeat fragments are the sequence having the sequence identity of more than 90% with SEQ ID NO: 29 and the sequence having the sequence identity of more than 90% with SEQ ID NO: 30 respectively, or the sequences of the two short inverted repeat fragments are the sequence having the sequence identity of more than 90% with SEQ ID NO: 31 and the sequence having the sequence identity of more than 90% with SEQ ID NO: 32 respectively.

Preferably, the sequence identity of more than 90% means that one base inconsistency exists between the sequences. In the sense strand, one inconsistent base is positioned at position 19 of the sense strand, and in the antisense strand, one inconsistent base is positioned at position 1 of the antisense strand.

More preferably, the nucleic acid is the plasmid inserted with the nucleic acid fragment encoding short hairpin ribonucleic acid. The plasmid expresses the short hairpin ribonucleic acid, and the nucleic acid fragment encoding the short hairpin ribonucleic acid comprises two short inverted repeat fragments and the loop fragment positioned between the two short inverted repeat fragments; and the sequences of the two short inverted repeat fragments are SEQ ID NO: 17 and SEQ ID NO: 18 respectively, or the sequences of the two short inverted repeat fragments are SEQ ID NO: 19 and SEQ ID NO: 20 respectively, or the sequences of the two short inverted repeat fragments are SEQ ID NO: 21 and SEQ ID NO: 22 respectively, or the sequences of the two short inverted repeat fragments are SEQ ID NO: 23 and SEQ ID NO: 24 respectively, or the sequences of the two short inverted repeat fragments are SEQ ID NO: 25 and SEQ ID NO: 26 respectively, or the sequences of the two short inverted repeat fragments are SEQ ID NO: 27 and SEQ ID NO: 28 respectively, or the sequences of the two short inverted repeat fragments are SEQ ID NO: 29 and SEQ ID NO: 30 respectively, or the sequences of the two short inverted repeat fragments are SEQ ID NO: 31 and SEQ ID NO: 32 respectively, or the sequences of the two short inverted repeat fragments are SEQ ID NO: 101 and SEQ ID NO: 102 respectively, or the sequences of the two short inverted repeat fragments are SEQ ID NO: 103 and SEQ ID NO: 104 respectively, or the sequences of the two short inverted repeat fragments are SEQ ID NO: 105 and SEQ ID NO: 106 respectively, or the sequences of the two short inverted repeat fragments are SEQ ID NO: 107 and SEQ ID NO: 108 respectively, or the sequences of the two short inverted repeat fragments are SEQ ID NO: 109 and SEQ ID NO: 110 respectively, or the sequences of the two short inverted repeat fragments are SEQ ID NO: 111 and SEQ ID NO: 112 respectively, or the sequences of the two short inverted repeat fragments are SEQ ID NO: 113 and SEQ ID NO: 114 respectively, or the sequences of the two short inverted repeat fragments are SEQ ID NO: 115 and SEQ ID NO: 116 respectively, or the sequences of the two short inverted repeat fragments are SEQ ID NO: 117 and SEQ ID NO: 118 respectively. In this case, the SEQ ID NO: 101, the SEQ ID NO: 103 and the SEQ ID NO: 105 are the sequences having the sequence identity of 90% with the SEQ ID NO: 17 respectively; the SEQ ID NO: 102, the SEQ ID NO: 104 and the SEQ ID NO: 106 are the sequences having the sequence identity of 90% with the SEQ ID NO: 18 respectively; the SEQ ID NO: 107, the SEQ ID NO: 109 and the SEQ ID NO: 111 are the sequences having the sequence identity of 90% with the SEQ ID NO: 21 respectively; the SEQ ID NO: 108, the SEQ ID NO: 110 and the SEQ ID NO: 112 are the sequences having the sequence identity of 90% with the SEQ ID NO: 22 respectively; the SEQ ID NO: 113, the SEQ ID NO: 115 and the SEQ ID NO: 117 are the sequences having the sequence identity of 90% with the SEQ ID NO: 25 respectively; and the SEQ ID NO: 114, the SEQ ID NO: 116 and the SEQ ID NO: 118 are the sequences having the sequence identity of 90% with the SEQ ID NO: 26 respectively.

In this case, for the two short inverted repeat fragments in the same shRNA, one is the sense short inverted repeat fragment corresponding to the sense strand of the siRNA, and the other one is the antisense short inverted repeat fragments corresponding to the antisense strand of the siRNA.

In this case, the sequence of the plasmid can include an empty vector sequence for expressing the shRNA and the sequence of the shRNA, and can further comprise other auxiliary sequences. In the case where the sequence of the shRNA to be expressed is clear, those skilled in the art can select, design, synthesize and/or use the plasmid through the conventional method to express the shRNA. For example, an empty vector for expressing the shRNA can be an empty vector product (the vectors numbered 1-8 can be used) purchased from a pGenesil series of Wuhan Genesil Company.

In this case, the loop fragment is used for forming a short hairpin structure of the shRNA with the two short inverted repeat fragments without damaging the function of the shRNA, the loop fragment can be a conventional choice in construction of the shRNA, such as the loop fragment mentioned in literature (Wang L, Mu F Y. A Web based design center for vector based siRNA and siRNA cassette Bioinformatics, 2004, 20 (11): 1818-1820), and such as SEQ ID NO: 81 (i.e., 5′-TCAAGAGA-3′). In this case, the sequence of the plasmid can also comprise an upstream transcriptional promoter sequence of the shRNA sequence (such as an RNA polymerase III promoter sequence, such as an H1 promoter or a U6 promoter) and a downstream transcriptional terminator sequence of the shRNA sequence (such as 5-6 continuous T). The sequence of the plasmid can further comprise a restriction enzyme cutting site to facilitate the molecular biological operation against the plasmid, such as enzyme cutting cloning and/or enzyme cutting identification and the like.

The invention further provides a pharmaceutical composition, which contains the nucleic acid as described above and a pharmaceutically acceptable carrier. The pharmaceutical composition can be prepared from the nucleic acid and the pharmaceutically acceptable carrier through the conventional method. For example, the pharmaceutical composition can be an injection. The injection can be used for subcutaneous, intramuscular or intravenous injection.

According to the pharmaceutical composition of the invention, there are no special requirements on the amount of the nucleic acid and the pharmaceutically acceptable carrier. Generally, relative to one part by weight of the nucleic acid, the content of the pharmaceutically acceptable carrier is 1-100000 parts by weight.

According to the pharmaceutical composition of the invention, wherein the pharmaceutically acceptable carrier can include various carrier which are conventionally adopted in the art, for example, the pharmaceutically acceptable carrier can include at least one of a pH value buffer solution, a protective agent and an osmosis pressure regulating agent. The pH value buffer solution can be a tris (hydroxymethyl) aminomethane hydrochloride buffer solution with the pH value of 7.5-8.5 and/or a phosphate buffer solution with the pH of 5.5-8.5, preferably the phosphate buffer solution with the pH of 5.5-8.5. The protective agent can be at least one of inositol, sorbitol and sucrose. By taking the total weight of the pharmaceutical composition as the reference, the content of the protective agent can be 0.01-30% by weight. The osmosis pressure regulating agent can be sodium chloride and/or potassium chloride. The content of the osmosis pressure regulating agent enables the osmosis pressure of the pharmaceutical composition to be 200-700 mOsmol/kg. According to the required osmosis pressure, those skilled in the art can determine the content of the osmosis pressure regulating agent.

According to a preferred embodiment of the invention, the pharmaceutically acceptable carrier is the vector covalently linking a liposome and bone-targeted molecules (a bone-targeted delivery system for treatment of osteogenesis based on the nucleic acid of the invention (namely the pharmaceutical composition of the invention) can be obtained by referring to a method recorded in CN102824647A).

In this case, the molar ratio of the part of the bone-targeted molecules to the part of the liposome is preferably (2-10): 100.

According to the pharmaceutical composition in the preferred embodiment of the invention, the molar ratio of the nucleic acid to the part of the liposome is preferably (5-10): 1, wherein the molar amount of the nucleic acid is calculated by element P, and the molar amount of the part of the liposome is calculated by element N.

More preferably, the liposome contains 1, 2-dioleoyl-3-trimethylammonium-propane (DOTAP), dioleoyl phosphatidylethanolamine (DOPE), cholesterol (Chol), distearoyl phosphoethanolamine-methoxypolyethylene glycol 2000 (N-(carbonyl-polyethylene glycol 2000)-1, 2-distearoyl-SN-glycero-3-phosphorylethanolamine, DSPE-mPEG2000) and distearoyl phosphoethanolamine-polyethylene glycol 2000-maleimide (N-(carbonyl-polyethylene glycol 2000)-1, 2-distearoyl-SN-glycero-3-phosphorylethanolamine-maleimide, DSPE-PEG2000-MAL). The molar ratio of the above various substances is preferably (20-25): (6-8): (15-20): (1-2):1.

More preferably, the bone-targeted molecules are a polypeptide with an amino acid sequence as shown in SEQ ID NO: 82.

In the above preferred pharmaceutically acceptable carrier, mercapto at the tail end of the bone-targeted molecules can directly react with a maleimide group of DSPE-PEG2000-MAL of the liposome, so as to complete covalent linking of the bone-targeted molecules and the liposome and directly connect the bone-targeted molecules on the surface of the liposome.

The using dosage of the pharmaceutical composition of the invention can be the conventional dosage in the art, and the dosage can be determined according to various parameters, in particular to age, body weight and gender of a subject. For example, for female C57BL/6J mice which are 3-4 months old and have a body weight of 25-30 g, based on the amount of the nucleic acid in the pharmaceutical composition, the using amount of the pharmaceutical composition can be 0.01-100 mg/kg of the body weight, preferably 1-10 mg/kg of the body weight. The invention further provides use of the nucleic acid as described above in preparation of a pharmaceutical composition for treating and/or preventing diseases related to the abnormal expression of CKIP-1 gene. In the pharmaceutical composition for treating and/or preventing the diseases related to the abnormal expression of CKIP-1 gene, the nucleic acid as described above mainly plays a role through the RNAi mechanism. Preferably, the diseases related to the abnormal expression of CKIP-1 gene include at least one of osteoporosis, osteoporotic fracture, fracture healing retardation, bone necrosis, degenerative arthritis and rheumatoid arthritis late-stage bone destruction.

The invention further provides a method for treating and/or preventing diseases related to the abnormal expression of CKIP-1 gene, the method comprising performing administration on a patient by using the nucleic acid and/or the pharmaceutical composition. The diseases related to the abnormal expression of CKIP-1 gene preferably include at least one of osteoporosis, osteoporotic fracture, fracture healing retardation, bone necrosis, degenerative arthritis and rheumatoid arthritis late-stage bone destruction.

In addition, the invention further provides a method for inhibiting the expression of CKIP-1 gene in cells, the method comprising introducing the nucleic acid and/or the pharmaceutical composition into the cells. The cells preferably are osteoblast-like cells.

The invention will be described in detail through the following embodiments. Unless particularly indicated, reagents and culture media used in the invention are commercially available commodities, and nucleic acid electrophoresis and other operations used in the invention are performed according to conventional protocols. In the following embodiments, all animal study procedures were conducted at Animal Center of Institute for Advancing Translational Medicine in Bone & Joint Diseases (TMBJ) in Hong Kong Baptist University and Laboratory Animal Service Center in Prince of Wales Hospital and approved by Ethics Committee in Hong Kong Baptist University (No. HASC/12-13/0032) and Animal Experimentation Ethics Committee in Chinese University of Hong Kong (No. 09/074/MIS).

Human osteoblast-like cells (hFOB1.19), rhesus osteoblast-like cells (isolated from cancellous iliac bone), rat osteoblast-like cells (UMR106), and mouse osteoblastic-like cells (MC3T3-E1) purchased from HOUBIO TECH Co., Ltd. Hong Kong, were cultured in DMEM medium (Gibco), containing 10% of fetal bovine serum (FBS, Gibco) and antibiotics (PSN, Gibco), and were incubated at 37° C. in a 5% CO₂/95% air humidified atmosphere. After the cell confluence reaches 70-80%, the cells were cultured in mineralization medium containing 10 mM β-glycerophosphate (Sigma) and 50 μm/ml ascorbic acid (Sigma).

When the cells were transfected by synthesized nucleic acid or non-specific siRNA (synthesized by Suzhou Ribo Life Science Co., Ltd.) in the preparation embodiment, Lipofectamine™ 2000 (Invitrogen) was used, and the specific operation parameters were as described in the manual provided by the manufacturer.

All data was represented by “mean values”, which was analyzed by one way analysis of variance (ANOVA) with a post hoc test to determine group differences in the study parameters using a statistical software program (SPSS version 13.0, SPSS, Chicago, Ill., USA).

### Preparation Embodiment 1

siRNA listed in Table 1 was obtained by an existing solid-phase synthesis method.

TABLE 1

| No. of | | Nucleotide | Sequence |
|---|---|---|---|
| siRNA | | sequence | No. |
| siRNA-1 | sense | 5′-CCUCUUGUGCUGAGAGCUUdTdT-3′ | SEQ ID NO: |
| | strand | | 1 |
| | antisense | 5′-AAGCUCUCAGCACAAGAGGdTdT-3′ | SEQ ID NO: |
| | strand | | 2 |
| siRNA-1A | sense | 5′-CCUCUUGUGCUGAGAGCUAdTdT-3′ | SEQ ID NO: |
| | strand | | 83 |
| | antisense | 5′-UAGCUCUCAGCACAAGAGGdTdT-3′ | SEQ ID NO: |
| | strand | | 84 |
| siRNA-1G | sense | 5′-CCUCUUGUGCUGAGAGCUGdTdT-3′ | SEQ ID NO: |
| | strand | | 85 |
| | antisense | 5′-CAGCUCUCAGCACAAGAGGdTdT-3′ | SEQ ID NO: |
| | strand | | 86 |
| siRNA-1C | sense | 5′-CCUCUUGUGCUGAGAGCUCdTdT-3′ | NO: SEQ ID |
| | strand | | 87 |
| | antisense | 5′-GAGCUCUCAGCACAAGAGGdTdT-3′ | SEQ ID NO: |
| | strand | | 88 |
| siRNA-2 | sense | 5′-UGAGAGACCUGUACAGACAdTdT-3′ | SEQ ID NO: |
| | strand | | 3 |
| | antisense | 5′-UGUCUGUACAGGUCUCUCAdTdT-3′ | SEQ ID NO: |
| | strand | | 4 |
| siRNA-3 | sense | 5′-CCUGAGUGACUAUGAGAAGdTdT-3′ | SEQ ID NO: |
| | strand | | 5 |
| | antisense | 5′-CUUCUCAUAGUCACUCAGGdTdT-3′ | SEQ ID NO: |
| | strand | | 6 |
| siRNA-3A | sense | 5′-CCUGAGUGACUAUGAGAAAdTdT-3′ | SEQ ID NO: |
| | strand | | 89 |
| | antisense | 5′-UUUCUCAUAGUCACUCAGGdTdT-3′ | SEQ ID NO: |
| | strand | | 90 |
| siRNA-3U | sense | 5′-CCUGAGUGACUAUGAGAAUdTdT-3′ | SEQ ID NO: |
| | strand | | 91 |
| | antisense | 5′-AUUCUCAUAGUCACUCAGGdTdT-3′ | SEQ ID NO: |
| | strand | | 92 |
| siRNA-3C | sense | 5′-CCUGAGUGACUAUGAGAACdTdT-3′ | SEQ ID NO: |
| | strand | | 93 |
| | antisense | 5′-GUUCUCAUAGUCACUCAGGdTdT-3′ | SEQ ID NO: |
| | strand | | 94 |
| siRNA-4 | sense | 5′-CCGGAAAUUCUGCGGGAAAdTdT-3′ | SEQ ID NO: |
| | strand | | 7 |
| | antisense | 5′-UUUCCCGCAGAAUUUCCGGdTdT-3′ | SEQ ID NO: |
| | strand | | 8 |
| siRNA-5 | sense | 5′-GGAUGAGGUCACCGUUGAGdTdT-3′ | SEQ ID NO: |
| | strand | | 9 |
| | antisense | 5′-CUCAACGGUGACCUCAUCCdTdT-3′ | SEQ ID NO: |
| | strand | | |
| siRNA-5A | sense | 5′-GGAUGAGGUCACCGUUGAAdTdT-3′ | SEQ ID NO: |
| | strand | | 95 |
| | antisense | 5′-UUCAACGGUGACCUCAUCCdTdT-3′ | SEQ ID NO: |
| | strand | | 96 |
| siRNA-5U | sense | 5′-GGAUGAGGUCACCGUUGAUdTdT-3′ | SEQ ID NO: |
| | strand | | 97 |
| | antisense | 5′-AUCAACGGUGACCUCAUCCdTdT-3′ | SEQ ID NO: |
| | strand | | 98 |
| siRNA-5C | sense | 5′-GGAUGAGGUCACCGUUGACdTdT-3′ | SEQ ID NO: |
| | strand | | 99 |
| | antisense | 5′-GUCAACGGUGACCUCAUCCdTdT-3′ | SEQ ID NO: |
| | strand | | 100 |
| siRNA-6 | sense | 5′-GUGCUGAGAGCUUUCGGGUdTdT-3′ | SEQ ID NO: |
| | strand | | 11 |
| | antisense | 5′-ACCCGAAAGCUCUCAGCACdTdT-3′ | SEQ ID NO: |
| | strand | | 12 |
| siRNA-7 | sense | 5′-GGUCGGCUGGGUCCGGAAAdTdT-3′ | SEQ ID NO: |
| | strand | | 13 |
| | antisense | 5′-UUUCCGGACCCAGCCGACCdTdT-3′ | SEQ ID NO: |
| | strand | | 14 |
| siRNA-8 | sense | 5′-ACCGCUAUGUGGUGCUGAAdTdT-3′ | SEQ ID NO: |
| | strand | | 15 |
| | antisense | 5′-UUCAGCACCACAUAGCGGUdTdT-3′ | SEQ ID NO: |
| | strand | | 16 |
| siRNA-NC | sense | 5′-UUCUCCGAACGUGUCACGUdTdT-3′ | SEQ ID NO: |
| | strand | | 41 |
| | antisense | 5′-ACGUGACACGUUCGGAGAAdTdT-3′ | SEQ ID NO: |
| | strand | | 42 |

### Preparation Embodiment 2

Modified siRNA listed in Table 2 was obtained by an existing solid-phase synthesis method, namely 2′-hydroxy groups of all nucleotide groups containing uracil bases or cytosine bases in the sense strand were modified by methoxy group, and 3′ ends of the sense strand and the antisense strand were connected with dTdT, respectively. The modified siRNA were referred to as m-siRNA, which were m-siRNA-1, m-siRNA-2, m-siRNA-3, m-siRNA-4, m-siRNA-5, m-siRNA-6, m-siRNA-7, m-siRNA-8 and modified non-specific siRNA (m-siRNA-NC), respectively. In this case, (OM) represented that the 2′ hydroxy group of the nucleotide group on the left was modified by the methoxy group.

The specific chemical modification schemes were as shown in Table 2:

TABLE 2

| | | | Corresponding |
|---|---|---|---|
| No. of | | | sequence |
| siRNA | | Nucleotide sequence | No. |
| m-siRNA-1 | sense | 5′-C (OM)C (OM)U (OM)C (OM)U | SEQ ID NO: 1 |
| | strand | (OM)U (OM)GU (OM)GC (OM)U | |
| | | (OM)GAGAGC (OM)U (OM)U | |
| | | (OM)dTdT-3′ | |
| | antisense | 5′-AAGCUCUCAGCACAAGAGGdTdT-3′ | SEQ ID NO: 2 |
| | strand | | |
| m-siRNA-1A | sense | 5′-C (OM)C (OM)U (OM)C (OM)U | SEQ ID NO: 83 |
| | strand | (OM)U (OM)GU (OM)GC (OM)U | |
| | | (OM)GAGAGC (OM)U (OM)AdTdT-3′ | |
| | antisense | 5′-UAGCUCUCAGCACAAGAGGdTdT-3′ | SEQ ID NO: 84 |
| | strand | | |
| m-siRNA-1G | sense | 5′-C (OM)C (OM)U (OM)C (OM)U | SEQ ID NO: 85 |
| | strand | (OM)U (OM)GU (OM)GC (OM)U | |
| | | (OM)GAGAGC (OM)U (OM)GdTdT-3′ | |
| | antisense | 5′-CAGCUCUCAGCACAAGAGGdTdT-3′ | SEQ ID NO: 86 |
| | strand | | |
| m-siRNA-1C | sense | 5′-C (OM)C (OM)U (OM)C (OM)U | SEQ ID NO: 87 |
| | strand | (OM)U (OM)GU (OM)GC (OM)U | |
| | | (OM)GAGAGC (OM)U (OM)CdTdT-3′ | |
| | antisense | 5′-GAGCUCUCAGCACAAGAGGdTdT-3′ | SEQ ID NO: 88 |
| | strand | | |
| m-siRNA-2 | sense | 5′-U (OM)GAGAGAC (OM)C (OM)U | SEQ ID NO: 3 |
| | strand | (OM)GU (OM)AC (OM)AGAC | |
| | | (OM)AdTdT-3′ | |
| | antisense | 5′-UGUCUGUACAGGUCUCUCAdTdT-3′ | SEQ ID NO: 4 |
| | strand | | |
| m-siRNA-3 | sense | 5′-C (OM)C (OM)U (OM)GAGU | SEQ ID NO: 5 |
| | strand | (OM)GAC (OM)U (OM)AU | |
| | | (OM)GAGAAGdTdT-3′ | |
| | antisense | 5′-CUUCUCAUAGUCACUCAGGdTdT-3′ | SEQ ID NO: 6 |
| | strand | | |
| m-siRNA-3A | sense | 5′-C(OM)C (OM)U (OM)GAGU | SEQ ID NO: 89 |
| | strand | (OM)GAC (OM)U (OM)AU | |
| | | (OM)GAGAAAdTdT-3′ | |
| | antisense | 5′-UUUCUCAUAGUCACUCAGGdTdT-3′ | SEQ ID NO: 90 |
| | strand | | |
| m-siRNA-3U | sense | 5′-C(OM)C(OM)U(OM)GAGU | SEQ ID NO: 91 |
| | strand | (OM)GAC (OM)U (OM)AU | |
| | | (OM)GAGAAU (OM)dTdT-3′ | |
| | antisense | 5′-AUUCUCAUAGUCACUCAGGdTdT-3′ | SEQ ID NO: 92 |
| | strand | | |
| m-siRNA-3C | sense | 5′-C (OM)C (OM)U (OM)GAGU | SEQ ID NO: 93 |
| | strand | (OM)GAC (OM)U (OM)AU | |
| | | (OM)GAGAACdTdT-3′ | |
| | antisense | 5′-GUUCUCAUAGUCACUCAGGdTdT-3′ | SEQ ID NO: 94 |
| | strand | | |
| m-siRNA-4 | sense | 5′-C (OM)C (OM)GGAAAU (OM)U | SEQ ID NO: 7 |
| | strand | (OM)C (OM)U (OM)GC | |
| | | (OM)GGGAAAdTdT-3′ | |
| | antisense | 5′-UUUCCCGCAGAAUUUCCGGdTdT-3′ | SEQ ID NO: 8 |
| | strand | | |
| m-siRNA-5 | sense | 5′-GGAU (OM)GAGGU (OM)C | SEQ ID NO: 9 |
| | strand | (OM)AC (OM)C (OM)GU (OM)U | |
| | | (OM)GAGdTdT-3′ | |
| | antisense | 5′-CUCAACGGUGACCUCAUCCdTdT-3′ | SEQ ID NO: 10 |
| | strand | | |
| m-siRNA-5A | sense | 5′-GGAU (OM)GAGGU (OM)C | SEQ ID NO: 95 |
| | strand | (OM)AC (OM)C (OM)GU (OM)U | |
| | | (OM)GAAdTdT-3′ | |
| | antisense | 5′-UUCAACGGUGACCUCAUCCdTdT-3′ | SEQ ID NO: 96 |
| | strand | | |
| m-siRNA-5U | sense | 5′-GGAU (OM)GAGG (OM)C | SEQ ID NO: 97 |
| | strand | (OM)AC (OM)C (OM)GU (OM)U | |
| | | (OM)GAU (OM)dTdT-3′ | |
| | antisense | 5′-AUCAACGGUGACCUCAUCCdTdT-3′ | SEQ ID NO: 98 |
| | strand | | |
| m-siRNA-5C | sense | 5′-GGAU (OM)GAGGU (OM)C | SEQ ID NO: 99 |
| | strand | (OM)AC (OM)C (OM)GU (OM)U | |
| | | (OM)GACdTdT-3′ | |
| | antisense | 5′-GUCAACGGUGACCUCAUCCdTdT-3′ | SEQ ID NO: 100 |
| | strand | | |
| m-siRNA-6 | sense | 5′-GU (OM)GC (OM)U (OM)GAGAGC | SEQ ID NO: 11 |
| | strand | (OM)U (OM)U (OM)U (OM)C | |
| | | (OM)GGGU (OM)dTdT-3′ | |
| | antisense | 5′-ACCCGAAAGCUCUCAGCACdTdT-3′ | SEQ ID NO: 12 |
| | strand | | |
| m-siRNA-7 | sense | 5′-GGU (OM)C (OM)GGC(OM)U | SEQ ID NO: 13 |
| | strand | (OM)GGGU (OM)C (OM)C | |
| | | (OM)GGAAAdTdT-3′ | |
| | antisense | 5′-UUUCCGGACCCAGCCGACCdTdT-3′ | SEQ ID NO: 14 |
| | strand | | |
| m-siRNA-8 | sense | 5′-AC (OM)C (OM)GC (OM)U | SEQ ID NO: 15 |
| | strand | (OM)AU (OM)GU (OM)GGU (OM)GC | |
| | | (OM)U (OM)GAAdTdT-3′ | |
| | antisense | 5′-UUCAGCACCACAUAGCGGUdTdT-3′ | SEQ ID NO: 16 |
| | strand | | |
| m-siRNA-NC | sense | 5′-U (OM)U (OM)C (OM)U (OM)C | SEQ ID NO: 41 |
| | strand | (OM)C (OM)GAAC (OM)GU (OM)GU | |
| | | (OM)C (OM)AC (OM)GU (OM)dTdT-3′ | |
| | antisense | 5′-ACGUGACACGUUCGGAGAAdTdT-3′ | SEQ ID NO: 42 |
| | strand | | |

The above modified siRNA was formed after annealing of the sense strand and the antisense strand in equal moles.

### Preparation Embodiment 3

A plasmid expressing shRNA listed in Table 3 was prepared by inserting a nucleic acid fragment encoding shRNA, which was formed by connecting a sense short inverted repeat fragment corresponding the sense strand of siRNA, a loop fragment (SEQ ID NO: 49, namely 5′-TCAAGAGA-3′) and an antisense short inverted repeat fragment corresponding to the antisense strand of siRNA in series, in an empty vector with the number of pGenesil-1 purchased from Wuhan Genesil Company (the sequence of the empty vector was as shown in the description). The plasmids expressing the shRNA were referred to as shRNA (p), which were shRNA (p)-1, shRNA (p)-2, shRNA (p)-3, shRNA (p)-4, shRNA (p)-5, shRNA (p)-6, shRNA (p)-′7, shRNA (p)-8 and shRNA (p)-NC expressing the non-specific siRNA sequence, respectively.

TABLE 3

| No. of | | Nucleotide sequence of short | |
|---|---|---|---|
| siRNA | | inverted repeat fragment | Sequence No. |
| shRNA (p)-1 | sense | 5′-CCTCTTGTGCTGAGAGCTT-3′ | SEQ ID NO: 17 |
| | strand | | |
| | antisense | 5′-AAGCTCTCAGCACAAGAGG-3′ | SEQ ID NO: 18 |
| | strand | | |
| shRNA (p)-1A | sense | 5′-CCTCTTGTGCTGAGAGCTA-3′ | SEQ ID NO: 101 |
| | strand | | |
| | antisense | 5′-TAGCTCTCAGCACAAGAGG-3′ | SEQ ID NO: 102 |
| | strand | | |
| shRNA (p)-1G | sense | 5′-CCTCTTGTGCTGAGAGCTG-3′ | SEQ ID NO: 103 |
| | strand | | |
| | antisense | 5′-CAGCTCTCAGCACAAGAGG-3′ | SEQ ID NO: 104 |
| | strand | | |
| shRNA (p)-1C | sense | 5′-CCTCTTGTGCTGAGAGCTC-3′ | SEQ ID NO: 105 |
| | strand | | |
| | antisense | 5′-GAGCTCTCAGCACAAGAGG-3′ | SEQ ID NO: 106 |
| | strand | | |
| shRNA (p)-2 | sense | 5′-TGAGAGACCTGTACAGACA-3′ | SEQ ID NO: 19 |
| | strand | | |
| | antisense | 5′-TGTCTGTACAGGTCTCTCA-3′ | SEQ ID NO: 20 |
| | strand | | |
| shRNA (p)-3 | sense | 5′-CCTGAGTGACTATGAGAAG-3′ | SEQ ID NO: 21 |
| | strand | | |
| | antisense | 5′-CTTCTCATAGTCACTCAGG-3′ | SEQ ID NO: 22 |
| | strand | | |
| shRNA (p)-3A | sense | 5′-CCTGAGTGACTATGAGAAA-3′ | SEQ ID NO: 107 |
| | strand | | |
| | antisense | 5′-TTTCTCATAGTCACTCAGG-3′ | SEQ ID NO: 108 |
| | strand | | |
| shRNA (p)-3T | sense | 5′-CCTGAGTGACTATGAGAAT-3′ | SEQ ID NO: 109 |
| | strand | | |
| | antisense | 5′-ATTCTCATAGTCACTCAGG-3′ | SEQ ID NO: 110 |
| | strand | | |
| shRNA (p)-3C | sense | 5′-CCTGAGTGACTATGAGAAC-3′ | SEQ ID NO: 111 |
| | strand | | |
| | antisense | 5′-GTTCTCATAGTCACTCAGG-3′ | SEQ ID NO: 112 |
| | strand | | |
| shRNA (p)-4 | sense | 5′-CCGGAAATTCTGCGGGAAA-3′ | SEQ ID NO: 23 |
| | strand | | |
| | antisense | 5′-TTTCCCGCAGAATTTCCGG-3′ | SEQ ID NO: 24 |
| | strand | | |
| shRNA (p)-5 | sense | 5′-GGATGAGGTCACCGTTGAG-3′ | SEQ ID NO: 25 |
| | strand | | |
| | antisense | 5′-CTCAACGGTGACCTCATCC-3′ | SEQ ID NO: 26 |
| | strand | | |
| shRNA (p)-5A | sense | 5′-GGATGAGGTCACCGTTGAA-3′ | SEQ ID NO: 113 |
| | strand | | |
| | antisense | 5′-TTCAACGGTGACCTCATCC-3′ | SEQ ID NO: 114 |
| | strand | | |
| shRNA (p)-5T | sense | 5′-GGATGAGGTCACCGTTGAT-3′ | SEQ ID NO: 115 |
| | strand | | |
| | antisense | 5′-ATCAACGGTGACCTCATCC-3′ | SEQ ID NO: 116 |
| | strand | | |
| shRNA (p)-5C | sense | 5′-GGATGAGGTCACCGTTGAC-3′ | SEQ ID NO: 117 |
| | strand | | |
| | antisense | 5′-GTCAACGGTGACCTCATCC-3′ | SEQ ID NO: 118 |
| | strand | | |
| shRNA (p)-6 | sense | 5′-GTGCTGAGAGCTTTCGGGT-3′ | SEQ ID NO: 27 |
| | strand | | |
| | antisense | 5′-ACCCGAAAGCTCTCAGCAC-3′ | SEQ ID NO: 28 |
| | strand | | |
| shRNA (p)-7 | sense | 5′-GGTCGGCTGGGTCCGGAAA-3′ | SEQ ID NO: 29 |
| | strand | | |
| | antisense | 5′-TTTCCGGACCCAGCCGACC-3′ | SEQ ID NO: 30 |
| | strand | | |
| shRNA (p)-8 | sense | 5′-ACCGCTATGTGGTGCTGAA-3′ | SEQ ID NO: 31 |
| | strand | | |
| | antisense | 5′-TTCAGCACCACATAGCGGT-3′ | SEQ ID NO: 32 |
| | strand | | |
| shRNA (p)-NC | sense | 5′-TTCTCCGAACGTGTCACGT-3′ | SEQ ID NO: 43 |
| | strand | | |
| | antisense | 5′-ACGTGACACGTTCGGAGAA-3′ | SEQ ID NO: 44 |
| | strand | | |

### Preparation Embodiment 4

The pharmaceutical composition of the invention was prepared according to the method in embodiment 3 of CN102824647A. The difference was that, the small nucleic acid drug was respectively replaced with the nucleic acids obtained in the preparation embodiments 1-3 (siRNA, m-siRNA and shRNA (p)), and the nucleic acid-free vector obtained according to the method in embodiment 3 of CN102824647A was named as a bone-targeted blank liposome (BTDS, bone targeting delivery system).

### Test Embodiment 1

The test embodiment was used for testing the in-vitro inhibition efficiency of the nucleic acids obtained in the preparation embodiments 1-3 against the expression of mRNA and proteins of CKIP-1 gene.

In vitro, the nucleic acids against CKIP-1 obtained in the preparation embodiments 1-3 were used for transfecting osteoblast-like cells of four organisms (human, rhesus, rats and mice) for being used as treatment groups (RNAi groups), a non-specific nucleic acid was used for transfecting the cells for being used as a control group (NC group), and a transfection reagent Lipofectamine™ 2000 (purchased from Invitrogen) was used for treating the cells alone for being used as a vehicle control group (VC group). Each group had four parallels (n=4) and the experiments were repeated at least four times. When the osteoblast-like cells of human, rhesus and mice were transfected, the final concentration of the nucleic acid was 40 nM; and when the osteoblast-like cells of rats were transfected, the final concentration of the nucleic acid was 80 nM. After 72 hours of transfection, the osteoblast-like cells of the various species were harvested and the expression of the mRNA and proteins of CKIP-1 was detected.

(1) Detection of CKIP-1 mRNA in Osteoblast-Like Cells

Real-time PCR was adopted for determining the expression level of CKIP-1 mRNA in each harvested osteoblast-like cells, and the specific operation was as follows: total RNA was extracted by using RNeasy Mini Kit (QIAGEN Company, Article number: Cat. 74106) according to the manufacturer's manual, and then reverse transcribed into cDNA, after that, a real-time PCR method was used for detecting the inhibition efficiency of the nucleic acid against the expression of the CKIP-1 mRNA in the osteoblast-like cells.

In the real-time PCR method, GAPDH gene was used as reference gene, and primers against CKIP-1 were used for detection, and the sequences of the primers were as shown in Table 4.

TABLE 4

| | | Upstream | Downstream |
|---|---|---|---|
| | Gene | primer | primer |
| Human | CKIP-1 | 5′-ACCCGAGCCAAGAACCGTAT-3′ | 5′-TGGAAGCCACAGCCATTAGG-3′ |
| | | (SEQ ID NO: 45) | (SEQ ID NO: 46) |
| | GAPDH | 5′-GGCATGGACTGTGGTCATGAG-3′ | 5′-TGCACCACCAACTGCTTAGC-3′ |
| | | (SEQ ID NO: 47) | (SEQ ID NO: 48) |
| Rhesus | CKIP-1 | 5′-TCACCCGAGCCAAGAACC-3′ | 5′-GGAAGCCACAGCCATTAGG-3′ |
| | | (SEQ ID NO: 49) | (SEQ ID NO: 50) |
| | GAPDH | 5′-TGACCTGCCGTCTGGAAA-3′ | 5′-GGGTGTCGCTGTTGAAGT-3′ |
| | | (SEQ ID NO: 51) | (SEQ ID NO: 52) |
| Rats | CKIP-1 | 5′-GAGCTTTCGGGTCGATCTGG-3′ | 5′-GGCTCCCTTGTCTGGTCTTT-3′ |
| | | (SEQ ID NO: 53) | (SEQ ID NO: 54) |
| | GAPDH | 5′-CAAGTTCAACGGCACAGTCA-3′ | 5′-CCATTTGATGTTAGCGGGAT-3′ |
| | | (SEQ ID NO: 55) | (SEQ ID NO: 56) |
| Mice | CKIP-1 | 5′-AACCGCTATGTGGTGCTGAA-3′ | 5′-CAGGGTGAACTTGCTGTGATT-3′ |
| | | (SEQ ID NO: 57) | (SEQ ID NO: 58) |
| | GAPDH | 5′-TGCACCACCAACTGCTTAG-3′ | 5′-GGATGCAGGGATGATGTTC-3′ |
| | | (SEQ ID NO: 59) | (SEQ ID NO: 60) |

In the real-time PCR method, the inhibition activity of the nucleic acid was calculated according to the following equation:

The inhibition activity of the nucleic acid=[1−(copy number of CKIP-1 of treatment group/copy number of GAPDH of treatment group)/(copy number of CKIP-1 of control group/copy number of GAPDH of control group)]×100%.

The results were as shown in Table 5.

TABLE 5
Inhibition rate against CKIP-1 mRNA in osteoblast-like cells (%)

| | | Human | Rhesus | Rats | Mice |
|---|---|---|---|---|---|
| NC | siRNA-NC | (0.00) | (0.00) | (0.00) | (0.00) |
| group | | | | | |
| VC | Lipo2000 | (0.45) | (−1.82) | (5.67) | (5.12) |
| group | | | | | |
| RNAi | siRNA-1 | 78.46 | 71.50 | 73.71 | 72.09 |
| group | siRNA-1A | 76.44 | 70.28 | 72.22 | 72.30 |
| | siRNA-1G | 70.32 | 68.88 | 67.56 | 65.45 |
| | siRNA-1C | 70.65 | 66.55 | 68.90 | 69.89 |
| | siRNA-2 | 76.78 | 79.10 | 81.75 | 45.75 |
| | siRNA-3 | 82.59 | 83.61 | 84.83 | 85.79 |
| | siRNA-3A | 81.06 | 82.98 | 82.34 | 80.86 |
| | siRNA-3U | 82.52 | 84.22 | 83.77 | 82.67 |
| | siRNA-3C | 81.56 | 80.12 | 80.67 | 81.32 |
| | siRNA-4 | (5.09) | (7.89) | (3.22) | (1.89) |
| | siRNA-5 | 75.64 | 73.00 | 74.33 | 74.33 |
| | siRNA-5A | 74.23 | 72.09 | 74.78 | 75.33 |
| | siRNA-5U | 75.67 | 72.98 | 75.68 | 77.80 |
| | siRNA-5C | 70.28 | 69.08 | 70.57 | 72.31 |
| | siRNA-6 | 67.16 | 74.46 | 63.21 | 54.21 |
| | siRNA-7 | 80.18 | 73.70 | 83.10 | 83.10 |
| | siRNA-8 | 72.84 | 78.28 | 80.26 | 81.16 |
| NC | m-siRNA-NC | (0.00) | (0.00) | (0.00) | (0.00) |
| group | | | | | |
| VC | Lipo2000 | (0.49) | (−2.68) | (4.94) | (5.39) |
| group | | | | | |
| RNAi | m-siRNA-1 | 81.29 | 73.88 | 73.71 | 73.80 |
| group | m-siRNA-1A | 80.03 | 74.32 | 74.30 | 72.89 |
| | m-siRNA-1G | 78.22 | 72.08 | 72.40 | 73.22 |
| | m-siRNA-1C | 77.98 | 73.80 | 73.01 | 74.00 |
| | m-siRNA-2 | 76.78 | 74.16 | 78.11 | 45.75 |
| | m-siRNA-3 | 88.24 | 88.18 | 90.28 | 88.43 |
| | m-siRNA-3A | 86.80 | 86.72 | 88.45 | 86.68 |
| | m-siRNA-3U | 88.59 | 87.72 | 88.04 | 86.02 |
| | m-siRNA-3C | 86.27 | 87.21 | 82.94 | 83.28 |
| | m-siRNA-4 | (3.04) | (6.82) | (−6.40) | (2.6) |
| | m-siRNA-5 | 75.65 | 80.81 | 75.21 | 74.60 |
| | m-siRNA-5A | 72.48 | 75.89 | 74.28 | 73.80 |
| | m-siRNA-5U | 76.08 | 78.96 | 77.28 | 75.38 |
| | m-siRNA-5C | 70.37 | 69.38 | 68.72 | 72.02 |
| | m-siRNA-6 | 67.16 | 72.13 | 70.46 | 54.21 |
| | m-siRNA-7 | 77.36 | 71.31 | 64.96 | 70.50 |
| | m-siRNA-8 | 72.84 | 76.30 | 65.75 | 66.76 |
| NC | shRNA | (0.00) | (0.00) | (0.00) | (0.00) |
| group | (p)-NC | | | | |
| VC | Lipo2000 | (0.38) | (1.67) | (5.34) | (4.85) |
| group | | | | | |
| RNAi | shRNA (p)-1 | 80.56 | 70.89 | 72.54 | 73.02 |
| group | shRNA (p)-2 | 74.58 | 78.56 | 79.68 | 46.32 |
| | shRNA (p)-3 | 83.21 | 84.25 | 82.78 | 81.14 |
| | shRNA (p)-4 | (6.88) | (9.02) | (5.56) | (2.27) |
| | shRNA (p)-5 | 72.02 | 78.25 | 74.33 | 72.22 |
| | shRNA (p)-6 | 63.64 | 72.54 | 64.21 | 55.27 |
| | shRNA (p)-7 | 82.03 | 72.14 | 80.12 | 81.22 |
| | shRNA (p)-8 | 73.56 | 77.54 | 76.38 | 78.68 |

Note:
VC, siRNA-1 to siRNA-8, m-siRNA-1 to m-siRNA-8 and shRNA (p)-1 to shRNA (p)-8 were respectively compared with siRNA-NC, m-siRNA-NC and shRNA (p)-NC in the same group.
( ) P > 0.05 represented that compared with the NC group, there was no statistically significant difference.

### (2) Immunoblotting Detection of Protein Level of CKIP-1 in Osteoblast-Like Cells

According to a method in literature (Molecular Cloning: A Laboratory Manual, Science Press, published in 2005), the immunoblotting detection was performed against the expression level of CKIP-1 proteins in the osteoblast-like cells. The CKIP-1 antibody used for immunoblotting detection was purchased from US Santa Cruz Biotechnology Company with the catalog number of sc-99218, and a reference antibody adopted a β-actin antibody which was purchased from US Santa Cruz Biotechnology Company with the catalog number of sc-47778.

In the immunoblotting method, the inhibition activity of the nucleic acid was calculated according to the following equation:

The inhibition activity of the nucleic acid=[1−(light intensity value of protein immunoblot strip of CKIP-1 of treatment group/light intensity value of protein immunoblot strip of β-actin of treatment group)/(light intensity value of protein immunoblot strip of CKIP-1 of control group/light intensity value of protein immunoblot strip of β-actin of control group)]×100%.

The results were as shown in Table 6.

TABLE 6
Inhibition rate against proteins of CKIP-1 in osteoblast-like cells (%)

| | | Human | Rhesus | Rats | Mice |
|---|---|---|---|---|---|
| NC group | siRNA-NC | (0.00) | (0.00) | (0.00) | (0.00) |
| VC group | Lipo2000 | (14.29) | (12.86) | (9.56) | (10.42) |
| RNAi group | siRNA-1 | 50.2 | 55.7 | 40.2 | 40.2 |
| | siRNA-2 | 49.8 | 52.6 | 43.2 | 40.2 |
| | siRNA-3 | 65.7 | 62.1 | 77.5 | 63.8 |
| | siRNA-4 | (−10.5) | (0.2) | (5.8) | (−10.3) |
| | siRNA-5 | 42.8 | 50.1 | 45.2 | 42.2 |
| | siRNA-6 | 40.2 | 48.2 | 55.6 | 30.6 |
| | siRNA-7 | 30.0 | 30.2 | 43.2 | 31.8 |
| | siRNA-8 | 20.4 | 28.4 | 41.6 | 30.5 |
| NC group | m-siRNA-NC | (0.00) | (0.00) | (0.00) | (0.00) |
| VC group | Lipo2000 | (16.01) | (10.56) | (7.88) | (8.59) |
| RNAi group | m-siRNA-1 | 59.56 | 68.46 | 63.38 | 56.18 |
| | m-siRNA-2 | 61.85 | 57.77 | 63.92 | 43.10 |
| | m-siRNA-3 | 84.43 | 86.98 | 82.11 | 81.70 |
| | m-siRNA-4 | (7.00) | (11.22) | (12.9) | (13.66) |
| | m-siRNA-5 | 62.50 | 59.89 | 63.08 | 64.47 |
| | m-siRNA-6 | 51.52 | 50.84 | 64.01 | 49.51 |
| | m-siRNA-7 | 61.79 | 53.09 | 52.29 | 59.49 |
| | m-siRNA-8 | 62.38 | 55.36 | 49.51 | 56.70 |
| NC group | shRNA (p)-NC | (0.00) | (0.00) | (0.00) | (0.00) |
| VC group | Lipo2000 | (13.98) | (8.76) | (10.34) | (7.28) |
| RNAi group | shRNA (p)-1 | 50.23 | 48.62 | 53.27 | 36.89 |
| | shRNA (p)-2 | 43.22 | 45.87 | 52.84 | 46.86 |
| | shRNA (p)-3 | 67.52 | 65.93 | 73.28 | 70.23 |
| | shRNA (p)-4 | (−7.2) | (10.58) | (−0.8) | (3.98) |
| | shRNA (p)-5 | 60.29 | 56.47 | 62.29 | 60.20 |
| | shRNA (p)-6 | 52.50 | 51.15 | 49.83 | 48.57 |
| | shRNA (p)-7 | 48.23 | 50.17 | 42.69 | 36.92 |
| | shRNA (p)-8 | 50.34 | 39.88 | 38.74 | 32.84 |

Note:
VC, siRNA-1 to siRNA-8, m-siRNA-1 to m-siRNA-8 and shRNA (p)-1 to shRNA (p)-8 were respectively compared with siRNA-NC, m-siRNA-NC and shRNA (p)-NC in the same group.
( ) P > 0.05 represented that compared with the NC group, there was no statistically significant difference.

### Test Embodiment 2

The test embodiment was used for analyzing in-vitro bone matrix mineralization deposition rate of the nucleic acid obtained in each of the preparation embodiments 1-3.

The nucleic acids against CKIP-1 obtained in the preparation embodiments 1-3 were used for transfecting osteoblast-like cells of four organisms (human, rhesus, rats and mice) for being used as treatment groups (RNAi groups), a non-specific nucleic acid was used for transfecting the cells for being used as a control group (NC group), and a transfection reagent Lipofectamine™ 2000 (purchased from Invitrogen) was used for treating the cells alone for being used as a vehicle control group (VC group). When the osteoblast-like cells of human, rhesus and mice were transfected, the final concentration of the nucleic acid was 40 nM; and when the osteoblast-like cells of rats were transfected, the final concentration of the nucleic acid was 80 nM. The frequency of transient transfection was once a week and each group had 4 parallels (n=4). At 7, 14 and 21 days after the first transfection, calcium deposition in the osteoblast-like cells of human and rhesus was respectively determined by calcium staining, at 48, 72 and 120 hours after the first transfection, calcium deposition in the osteoblast-like cells of rats was determined by calcium staining, at 7, 10 and 14 days after the first transfection, calcium deposition in the osteoblast-like cells of mice was determined by calcium staining (by using a Sigma Diagnostic Kit#587-A, and the specific operation was as described in the manufacturer's manual), and the results showed that at 21 days after the first transfection of the osteoblast-like cells of human and rhesus, at 120 hours after the first transfection of the osteoblast-like cells of rats and at 14 days after the first transfection of the osteoblast-like cells of mice, calcium deposition in the RNAi group was significantly higher than that in the VC group and the NC group, and the specific data was as shown in Table 7.

TABLE 7
Calcium deposition in osteoblast-like cells (ng/μg of proteins)

| | | Human | Rhesus | Rats | Mice |
|---|---|---|---|---|---|
| NC group | siRNA-NC | (23.4) | (39.8) | (61.6) | (62.3) |
| VC group | Lipo2000 | (30.4) | (43.1) | (60.7) | (58.7) |
| RNAi group | siRNA-1 | 40.1 | 56.7 | 113.7 | 96.9 |
| | siRNA-1A | 38.2 | 55.3 | 110.2 | 95.7 |
| | siRNA-1G | 36.7 | 54.7 | 98.6 | 96.2 |
| | siRNA-1C | 37.2 | 55.2 | 99.7 | 95.4 |
| | siRNA-2 | 34.4 | 47.2 | 85.6 | 70.3 |
| | siRNA-3 | 39.8 | 60.5 | 119.5 | 91.5 |
| | siRNA-3A | 38.9 | 58.2 | 113.4 | 88.6 |
| | siRNA-3U | 40.2 | 58.7 | 114.2 | 89.8 |
| | siRNA-3C | 38.2 | 56.4 | 111.3 | 86.6 |
| | siRNA-4 | (23.5) | (43.9) | (61.4) | (66.9) |
| | siRNA-5 | 45.9 | 51.3 | 103.2 | 76.5 |
| | siRNA-5A | 44.6 | 50.2 | 100.6 | 76.5 |
| | siRNA-5U | 44.2 | 50.8 | 100.8 | 78.2 |
| | siRNA-5C | 42.1 | 48.2 | 98.5 | 74.6 |
| | siRNA-6 | 32.3 | 46.9 | 98.0 | 84.9 |
| | siRNA-7 | 32.9 | 44.3 | 73.9 | 69.3 |
| | siRNA-8 | 38.7 | 44.2 | 69.2 | 69.9 |
| NC group | m-siRNA-NC | (27.8) | (47.5) | (67.5) | (66.5) |
| VC group | Lipo2000 | (29.0) | (44.8) | (63.3) | (50.8) |
| RNAi group | m-siRNA-1 | 46.5 | 60.8 | 120.6 | 102.5 |
| | m-siRNA-1A | 44.3 | 58.2 | 118.6 | 99.2 |
| | m-siRNA-1G | 40.8 | 53.2 | 117.3 | 96.5 |
| | m-siRNA-1C | 41.2 | 52.9 | 115.4 | 94.2 |
| | m-siRNA-2 | 38.5 | 50.4 | 98.5 | 79.3 |
| | m-siRNA-3 | 42.3 | 63.5 | 122.8 | 97.3 |
| | m-siRNA-3A | 40.8 | 60.2 | 118.7 | 96.5 |
| | m-siRNA-3U | 44.1 | 62.9 | 124.0 | 98.6 |
| | m-siRNA-3C | 38.8 | 59.2 | 116.4 | 92.4 |
| | m-siRNA-4 | (28.3) | (48.3) | (65.7) | (68.5) |
| | m-siRNA-5 | 49.5 | 54.3 | 128.4 | 91.9 |
| | m-siRNA-5A | 46.2 | 55.2 | 122.6 | 90.0 |
| | m-siRNA-5U | 47.2 | 52.7 | 126.8 | 89.6 |
| | m-siRNA-5C | 45.7 | 50.2 | 120.6 | 84.2 |
| | m-siRNA-6 | 36.4 | 50.3 | 110.5 | 89.5 |
| | m-siRNA-7 | 34.5 | 52.3 | 78.8 | 72.3 |
| | m-siRNA-8 | 41.5 | 49.9 | 70.3 | 70.5 |
| NC group | shRNA (p)-NC | (25.5) | (45.2) | (67.5) | (66.0) |
| VC group | Lipo2000 | (28.2) | (42.5) | (65.1) | (60.2) |
| RNAi group | shRNA (p)-1 | 39.6 | 58.1 | 116.5 | 91.6 |
| | shRNA (p)-2 | 30.2 | 50.9 | 88.2 | 80.5 |
| | shRNA (p)-3 | 39.5 | 60.9 | 108.3 | 90.5 |
| | shRNA (p)-4 | (22.5) | (45.2) | (65.4) | (66.1) |
| | shRNA (p)-5 | 42.1 | 52.0 | 109.5 | 87.6 |
| | shRNA (p)-6 | 32.4 | 50.6 | 106.4 | 80.3 |
| | shRNA (p)-7 | (29.4) | 50.9 | 74.3 | 74.9 |
| | shRNA (p)-8 | 36.1 | (45.5) | 70.2 | 72.1 |

Note:
VC, siRNA-1 to siRNA-8, m-siRNA-1 to m-siRNA-8 and shRNA (p)-1 to shRNA (p)-8 were respectively compared with siRNA-NC, m-siRNA-NC and shRNA (p)-NC in the same group.
( ) P > 0.05 represented that compared with the NC group, there was no statistically significant difference.

Calcium deposition is a key functional mineralization marker of mature osteoblasts in-vitro during osteoblastogenesis. Calcium deposition of the RNAi group was obviously higher than that in the VC group and the NC group, which suggested that the nucleic acid of the invention could promote differentiation from pre-osteoblast to mature osteoblast across the four species at the functional level.

### Test Embodiment 3

The test embodiment was used for testing the in-vivo influence of the pharmaceutical composition containing the nucleic acid obtained in the preparation embodiment 4 on the expression of CKIP-1 mRNA, osteoblast phenotype gene and biochemical markers.

In this assay, 960 6-month-old female Sprague-Dawley rats or 960 4-month-old female C57/BL mice were divided into nucleic acid treatment group (RNAi group, n=840), non-specific nucleic acid control group (NC group, n=60) and vehicle control group (VC group, n=60). As for rats or mice, each group had 20 animals. The pharmaceutical composition obtained in the preparation embodiment 4 was respectively injected into rats or mice in the RNAi group and NC group, and the animals in the VC group were only subject to BTDS injection. The injection dosage of the nucleic acid was 4 mg/kg in rats, and 7.5 mg/kg in mice. All the nucleic acid was labeled with FAM-fluorescence, and injected by intravenous injection of tail. Four rats or mice in each group were euthanized at day 0, 1, 3, 5 and 7 after injection, respectively. Then, bone marrow from bilateral femur was collected.

(1) Analysis of Expression of mRNA of Targeting Gene CKIP-1

The bone marrow of the euthanized rats or mice was collected, the intraosseous mRNA expression level of CKIP-1 was detected according to the method of “detection of CKIP-1 mRNA in osteoblast-like cells” in the test embodiment 1, and it was found that the in-vivo mRNA expression of CKIP-1 in the RNAi group was significantly reduced in comparison with the NC group or the VC group, and the expression could persist for 7 days. At 7 days after injection, all the rats or mice were euthanized, the bone marrow from bilateral femur was collected, the in-vivo inhibition efficiency against CKIP-1 mRNA was detected and the results were as shown in Table 8.

TABLE 8

| | | Inhibition rate | Inhibition rate |
|---|---|---|---|
| | | against CKIP-1 in | against CKIP-1 in |
| | | bone marrow | bone marrow |
| | | of rats (%) | of mice (%) |
| NC | siRNA-NC | (0.0) | (0.0) |
| group | | | |
| VC | BTDS | (3.4) | (6.0) |
| group | | | |
| RNAi | siRNA-1 | 68.3 | 55.3 |
| group | siRNA-1A | 66.2 | 54.2 |
| | siRNA-1G | 62.1 | 50.2 |
| | siRNA-1C | 62.4 | 52.5 |
| | siRNA-2 | 57.3 | 46.2 |
| | siRNA-3 | 70.1 | 59.9 |
| | siRNA-3A | 69.2 | 56.2 |
| | siRNA-3U | 71.2 | 58.9 |
| | siRNA-3C | 66.4 | 52.4 |
| | siRNA-4 | (2.3) | (0.6) |
| | siRNA-5 | 60.1 | 50.6 |
| | siRNA-5A | 58.4 | 48.8 |
| | siRNA-5U | 60.2 | 51.2 |
| | siRNA-5C | 55.2 | 46.2 |
| | siRNA-6 | 50.1 | 47.9 |
| | siRNA-7 | 56.3 | 43.2 |
| | siRNA-8 | 42.4 | 40.3 |
| NC | m-siRNA-NC | (0.0) | (0.0) |
| group | | | |
| VC | BTDS | (3.2) | (6.4) |
| group | | | |
| RNAi | m-siRNA-1 | 71.4 | 60.2 |
| group | m-siRNA-1A | 70.2 | 58.5 |
| | m-siRNA-1G | 68.5 | 52.3 |
| | m-siRNA-1C | 66.2 | 54.3 |
| | m-siRNA-2 | 60.1 | 50.2 |
| | m-siRNA-3 | 72.4 | 63.1 |
| | m-siRNA-3A | 70.2 | 58.9 |
| | m-siRNA-3U | 72.4 | 62.2 |
| | m-siRNA-3C | 68.9 | 54.2 |
| | m-siRNA-4 | (7.3) | (1.9) |
| | m-siRNA-5 | 66.2 | 56.9 |
| | m-siRNA-5A | 64.2 | 55.2 |
| | m-siRNA-5U | 66.4 | 57.0 |
| | m-siRNA-5C | 60.2 | 50.2 |
| | m-siRNA-6 | 53.2 | 52.7 |
| | m-siRNA-7 | 60.2 | 48.9 |
| | m-siRNA-8 | 50.3 | 52.4 |
| NC | shRNA (p)-NC | (0.0) | (0.0) |
| group | | | |
| VC | BTDS | (2.6) | (5.8) |
| group | | | |
| RNAi | shRNA (p)-1 | 70.1 | 58.1 |
| group | shRNA (p)-2 | 52.3 | 38.9 |
| | shRNA (p)-3 | 68.3 | 62.6 |
| | shRNA (p)-4 | (−1.6) | (7.3) |
| | shRNA (p)-5 | 56.3 | 53.9 |
| | shRNA (p)-6 | 48.2 | 50.1 |
| | shRNA (p)-7 | 56.2 | 49.3 |
| | shRNA (p)-8 | 46.2 | 48.7 |

Note:
VC, siRNA-1 to siRNA-8, m-siRNA-1 to m-siRNA-8 and shRNA (p)-1 to shRNA (p)-8 were respectively compared with siRNA-NC, m-siRNA-NC and shRNA (p)-NC in the same group.
( ) P > 0.05 represented that compared with the NC group, there was no statistically significant difference.

### (2) Analysis of Influence on Differentiation of Osteoblasts

The bone marrow of the euthanized rats or mice was collected, the time-course changes in mRNA expression levels of osteoblast phenotype genes, such as alkaline phosphatase (ALP), I type collagen (COL1), osteopontin (OPN), bone sialoprotein (BSP) and osteocalcin (OC) were detected in the bone marrow from bilateral femur, according to the method “detection of CKIP-1 mRNA in osteoblasts” in the test embodiment 1, and the used primers were as shown in Table 9. The results showed that, at day 3 after injection, the mRNA expression of ALP, COL1 and OPN in the RNAi group was significantly increased in comparison with the NC group and the VC group; and at day 5 after injection, the mRNA expression of BSP and OC in the RNAi group was significantly increased in comparison with the NC group and the VC group. The results were as shown in Tables 10-14, wherein ( ) P>0.05 represented that compared with the NC group, there was no statistically significant difference.

TABLE 9

| | Gene | Forward primer | Reverse primer |
|---|---|---|---|
| Rats | ALP | 5′-TGGACGGTGAACGGGAGAAC-3′ | 5′-GGACGCCGTGAAGCAGGTGA-3′ |
| | | (SEQ ID NO: 61) | (SEQ ID NO: 62) |
| | COL1 | 5′-CCTACAGCACGCTTGTGGAT-3′ | 5′-ATTGGGATGGAGGGAGTTTA-3′ |
| | | (SEQ ID NO: 63) | (SEQ ID NO: 64) |
| | OPN | 5′-AGAAACGGATGACTTTAAGCAAG | 5′-TCTCTGCATGGTCTCCATCGT-3′ |
| | | AA-3′ (SEQ ID NO: 65) | (SEQ ID NO: 66) |
| | BSP | 5′-AGAAAGAGCAGCACGGTTGA | 5′-CCCTCGTAGCCTTCATAGCC-3′ |
| | | (SEQ ID NO: 67) | (SEQ ID NO: 68) |
| | OC | 5′-CTCACTCTGCTGGCCCTGAC-3′ | 5′-CCTTACTGCCCTCCTGCTTG-3′ |
| | | (SEQ ID NO: 69) | (SEQ ID NO: 70) |
| | GAPDH | 5′-CAAGTTCAACGGCACAGTCA-3′ | 5′-CCATTTGATGTTAGCGGGAT-3′ |
| | | (SEQ ID NO: 55) | (SEQ ID NO: 56) |
| Mice | ALP | 5′-ATCTTTGGTCTGGCTCCCATG-3′ | 5′-TTTCCCGTTCACCGTCCAC-3′ |
| | | (SEQ ID NO: 71) | (SEQ ID NO: 72) |
| | COL1 | 5′-CCTGGTAAAGATGGTGCC-3′ | 5′-CACCAGGTTCACCTTTCGCACC-3′ |
| | | (SEQ ID NO: 73) | (SEQ ID NO: 74) |
| | OPN | 5′-ACACTTTCACTCCAATCGTCC-3′ | 5′-TGCCCTTTCCGTTGTTGTCC-3′ |
| | | (SEQ ID NO: 75) | (SEQ ID NO: 76) |
| | BSP | 5′-AAGCAGCACCGTTGAGTATGG-3′ | 5′-CCTTGTAGTAGCTGTATTCGTCC |
| | | (SEQ ID NO: 77) | TC-3′ (SEQ ID NO: 78) |
| | OC | 5′-GCAATAAGGTAGTGAACAGACTC | 5′-GTTTGTAGGCGGTCTTCAAGC-3′ |
| | | C-3′ (SEQ ID NO: 79) | (SEQ ID NO: 80) |
| | GAPDH | 5′-TGCACCACCAACTGCTTAG-3′ | 5′-GGATGCAGGGATGATGTTC-3′ |
| | | (SEQ ID NO: 59) | (SEQ ID NO: 60) |

TABLE 10

| | | Upregulation of | Upregulation of |
|---|---|---|---|
| | | ALP mRNA | ALP mRNA |
| | | in bone marrow | in bone marrow |
| | | of rats (%) | of mice (%) |
| NC | siRNA-NC | (0.0) | (0.0) |
| group | | | |
| VC | BTDS | (5.6) | (8.7) |
| group | | | |
| RNAi | siRNA-1 | 89.2 | 60.3 |
| group | siRNA-2 | 45.2 | 39.7 |
| | siRNA-3 | 121.2 | 57.4 |
| | siRNA-4 | (1.6) | (−3.5) |
| | siRNA-5 | 80.1 | 53.9 |
| | siRNA-6 | 32.1 | 43.2 |
| | siRNA-7 | 35.2 | 43.9 |
| | siRNA-8 | 42.1 | 38.2 |
| NC | m-siRNA-NC | (0.0) | (0.0) |
| group | | | |
| VC | BTDS | (−10.4) | (23.4) |
| group | | | |
| RNAi | m-siRNA-1 | 102.3 | 62.1 |
| group | m-siRNA-2 | 63.8 | 50.2 |
| | m-siRNA-3 | 121.9 | 59.1 |
| | m-siRNA-4 | (−2.5) | (5.7) |
| | m-siRNA-5 | 93.2 | 62.1 |
| | m-siRNA-6 | 37.5 | 50.2 |
| | m-siRNA-7 | 46.8 | 48.3 |
| | m-siRNA-8 | 50.1 | 45.2 |
| NC | shRNA (p)-NC | (0.0) | (0.0) |
| group | | | |
| VC | BTDS | (5.7) | (12.9) |
| group | | | |
| RNAi | shRNA (p)-1 | 86.3 | 50.2 |
| group | shRNA (p)-2 | 46.2 | 37.9 |
| | shRNA (p)-3 | 95.3 | 57.2 |
| | shRNA (p)-4 | (9.2) | (10.3) |
| | shRNA (p)-5 | 81.4 | 42.5 |
| | shRNA (p)-6 | 30.1 | 32.5 |
| | shRNA (p)-7 | 42.1 | 45.3 |
| | shRNA (p)-8 | 39.1 | 42.6 |

TABLE 11

| | | Upregulation of COL1 | Upregulation of COL1 |
|---|---|---|---|
| | | mRNA in bone | mRNA in bone |
| | | marrow of rats (%) | marrow of mice (%) |
| NC | siRNA-NC | (0.0) | (0.0) |
| group | | | |
| VC | BTD S | (7.2) | (−9.4) |
| group | | | |
| RNAi | siRNA-1 | 82.3 | 98.1 |
| group | siRNA-2 | 58.2 | 60.3 |
| | siRNA-3 | 90.3 | 112.8 |
| | siRNA-4 | (−3.8) | (12.7) |
| | siRNA-5 | 80.3 | 85.9 |
| | siRNA-6 | 51.3 | 63.1 |
| | siRNA-7 | 50.1 | 51.4 |
| | siRNA-8 | 38.2 | 32.5 |
| NC | m-siRNA-NC | (0.0) | (0.0) |
| group | | | |
| VC | BTDS | (9.2) | (−13.5) |
| group | | | |
| RNAi | m-siRNA-1 | 92.1 | 132.5 |
| group | m-siRNA-2 | 56.3 | 73.2 |
| | m-siRNA-3 | 99.1 | 152.5 |
| | m-siRNA-4 | (4.8) | (9.2) |
| | m-siRNA-5 | 87.3 | 98.8 |
| | m-siRNA-6 | 52.3 | 67.2 |
| | m-siRNA-7 | 49.6 | 56.9 |
| | m-siRNA-8 | 42.1 | 39.5 |
| NC | shRNA (p)-NC | (0.0) | (0.0) |
| group | | | |
| VC | BTDS | (10.3) | (−10.4) |
| group | | | |
| RNAi | shRNA (p)-1 | 80.5 | 99.2 |
| group | shRNA (p)-2 | 51.3 | 70.9 |
| | shRNA (p)-3 | 83.7 | 106.3 |
| | shRNA (p)-4 | (12.1) | (7.2) |
| | shRNA (p)-5 | 69.3 | 68.2 |
| | shRNA (p)-6 | 50.2 | 51.0 |
| | shRNA (p)-7 | 49.3 | 52.1 |
| | shRNA (p)-8 | 40.2 | 39.6 |

TABLE 12

| | | Upregulation of OPN | Upregulation of OPN |
|---|---|---|---|
| | | mRNA in bone | mRNA in bone |
| | | marrow of rats (%) | marrow of mice (%) |
| NC | siRNA-NC | (0.0) | (0.0) |
| group | | | |
| VC | BTDS | (−14.4) | (−12.3) |
| group | | | |
| RNAi | siRNA-1 | 93.5 | 90.5 |
| group | siRNA-2 | 65.3 | 63.7 |
| | siRNA-3 | 105.2 | 119.4 |
| | siRNA-4 | (5.2) | (12.9) |
| | siRNA-5 | 87.2 | 79.6 |
| | siRNA-6 | 63.4 | 72.3 |
| | siRNA-7 | 50.9 | 46.8 |
| | siRNA-8 | 49.8 | 40.3 |
| NC | m-siRNA-NC | (0.0) | (0.0) |
| group | | | |
| VC | BTDS | (−15.0) | (−17.6) |
| group | | | |
| RNAi | m-siRNA-1 | 102.5 | 97.5 |
| group | m-siRNA-2 | 72.1 | 69.3 |
| | m-siRNA-3 | 120.6 | 127.1 |
| | m-siRNA-4 | (13.2) | (17.4) |
| | m-siRNA-5 | 89.2 | 80.3 |
| | m-siRNA-6 | 62.1 | 68.3 |
| | m-siRNA-7 | 53.2 | 49.3 |
| | m-siRNA-8 | 50.2 | 38.6 |
| NC | shRNA (p)-NC | (0.0) | (0.0) |
| group | | | |
| VC | BTDS | (−10.8) | (−12.2) |
| group | | | |
| RNAi | shRNA (p)-1 | 90.3 | 87.7 |
| group | shRNA (p)-2 | 63.2 | 60.5 |
| | shRNA (p)-3 | 101.7 | 96.8 |
| | shRNA (p)-4 | (13.1) | (−12.9) |
| | shRNA (p)-5 | 83.4 | 79.8 |
| | shRNA (p)-6 | 60.2 | 58.3 |
| | shRNA (p)-7 | 49.6 | 50.1 |
| | shRNA (p)-8 | 49.4 | 41.6 |

TABLE 13

| | | Upregulation of BSP | Upregulation of BSP |
|---|---|---|---|
| | | mRNA in bone | mRNA in bone |
| | | marrow of rats (%) | marrow of mice (%) |
| NC | siRNA-NC | (0.0) | (0.0) |
| group | | | |
| VC | BTDS | (−5.0) | (8.5) |
| group | | | |
| RNAi | siRNA-1 | 74.3 | 49.2 |
| group | siRNA-2 | 50.2 | 40.4 |
| | siRNA-3 | 80.2 | 50.1 |
| | siRNA-4 | (−10.4) | (3.7) |
| | siRNA-5 | 63.8 | 42.9 |
| | siRNA-6 | 70.3 | 46.1 |
| | siRNA-7 | 36.2 | 40.3 |
| | siRNA-8 | 35.2 | 40.5 |
| NC | m-siRNA-NC | (0.0) | (0.0) |
| group | | | |
| VC | BTDS | (−5.8) | (9.2) |
| group | | | |
| RNAi | m-siRNA-1 | 90.2 | 53.2 |
| group | m-siRNA-2 | 61.2 | 54.8 |
| | m-siRNA-3 | 99.1 | 55.3 |
| | m-siRNA-4 | (9.3) | (2.5) |
| | m-siRNA-5 | 80.2 | 51.3 |
| | m-siRNA-6 | 72.7 | 49.6 |
| | m-siRNA-7 | 42.1 | 45.6 |
| | m-siRNA-8 | 40.3 | 39.5 |
| NC | shRNA (p)-NC | (0.0) | (0.0) |
| group | | | |
| VC | BTDS | (2.2) | (4.5) |
| group | | | |
| RNAi | shRNA (p)-1 | 85.2 | 49.8 |
| group | shRNA (p)-2 | 52.2 | 45.3 |
| | shRNA (p)-3 | 93.6 | 52.7 |
| | shRNA (p)-4 | (10.2) | (7.4) |
| | shRNA (p)-5 | 76.3 | 49.5 |
| | shRNA (p)-6 | 70.3 | 43.5 |
| | shRNA (p)-7 | 39.8 | 42.6 |
| | shRNA (p)-8 | 43.6 | 35.1 |

TABLE 14

| | | Upregulation of OC | Upregulation of OC |
|---|---|---|---|
| | | mRNA in bone | mRNA in bone |
| | | marrow of rats (%) | marrow of mice (%) |
| NC | siRNA-NC | (0.0) | (0.0) |
| group | | | |
| VC | BTDS | (−3.2) | (−2.0) |
| group | | | |
| RNAi | siRNA-1 | 53.2 | 45.2 |
| group | siRNA-2 | 51.1 | 42.9 |
| | siRNA-3 | 56.2 | 45.3 |
| | siRNA-4 | (3.6) | (7.2) |
| | siRNA-5 | 50.2 | 41.2 |
| | siRNA-6 | 42.6 | 38.3 |
| | siRNA-7 | 40.6 | 35.9 |
| | siRNA-8 | 37.8 | 36.2 |
| NC | m-siRNA-NC | (0.0) | (0.0) |
| group | | | |
| VC | BTDS | (−3.7) | (−2.1) |
| group | | | |
| RNAi | m-siRNA-1 | 55.3 | 47.2 |
| group | m-siRNA-2 | 43.5 | 40.7 |
| | m-siRNA-3 | 58.1 | 49.0 |
| | m-siRNA-4 | (−2.8) | (4.3) |
| | m-siRNA-5 | 50.2 | 41.8 |
| | m-siRNA-6 | 48.2 | 38.8 |
| | m-siRNA-7 | 40.3 | 42.1 |
| | m-siRNA-8 | 38.4 | 36.2 |
| NC | shRNA (p)-NC | (0.0) | (0.0) |
| group | | | |
| VC | BTDS | (−2.5) | (−1.8) |
| group | | | |
| RNAi | shRNA (p)-1 | 50.2 | 43.6 |
| group | shRNA (p)-2 | 48.2 | 42.7 |
| | shRNA (p)-3 | 51.8 | 48.5 |
| | shRNA (p)-4 | (−4.6) | (3.8) |
| | shRNA (p)-5 | 48.2 | 39.2 |
| | shRNA (p)-6 | 36.9 | 32.4 |
| | shRNA (p)-7 | 32.8 | 32.6 |
| | shRNA (p)-8 | 40.1 | 35.9 |

Note:
in Tables 10-14, VC, siRNA-1 to siRNA-8, m-siRNA-1 to m-siRNA-8 and shRNA (p)-1 to shRNA (p)-8 were respectively compared with siRNA-NC, m-siRNA-NC and shRNA (p)-NC in the same group.
( ) P > 0.05 represented that compared with the NC group, there was no statistically significant difference.

It was known to those skilled in the art that ALP, COL1A1 and OPN expressions appear at the early stage of osteoblast differentiation, BSP and OC expressions don't appear until osteoblast reaches more mature functional stage. The results of Tables 10-14 showed that, after injection, the nucleic acid of the invention could promote the expression of osteoblast phenotype genes, indicating that the nucleic acid of the invention could promote the differentiation of osteoblasts in vivo on the molecular level.

### (3) Analysis of Expression of Biochemical Markers

At day 0, 1, 3, 5 and 7 after injection and before euthanizing the rats and mice, heart blood and urine were collected respectively, the expression levels of serum bone formation marker PINP (rat/mouse PINP EIA kit, purchased from Immunodiagnostic Systems Company, Catalog No. AC33F1) and urine bone resorption marker DPD (DPD EIA kit, purchased from CUSABIO Company, Catalog No. CSB-E08400r (rats), CSB-E08401m (mice)) were detected by using the ELISA kits according to the manufacturer's manuals, wherein DPD was represented by relative level relative to Creatinine (Cr) The results showed that, at day 5 after injection, the level of serum PINP in the RNAi group was significantly increased in comparison with the NC group and the VC group, but the level of urine DPD did not change obviously, and the results were as shown in Table 15.

TABLE 15

| | | Rats | | Mice | |
|---|---|---|---|---|---|
| | | Level of | Level of | Level of | Level of |
| | | serum | urine DPD | serum | urine DPD |
| | | PINP | relative to Cr | PINP | relative to Cr |
| | | (μg/ml) | (nmol/mmol) | (μg/ml) | (nmol/mmol) |
| NC | siRNA-NC | 2.5 | 30.2 | 0.13 | 10.0 |
| group | | | | | |
| VC | BTDS | 2.5 | 32.2 | 0.12 | 10.5 |
| group | | | | | |
| RNAi | siRNA-1 | 3.4* | 36.1 | 0.20* | 10.3 |
| group | siRNA-2 | 2.8* | 32.6 | 0.18* | 13.1 |
| | siRNA-3 | 3.5* | 35.3 | 0.22* | 12.5 |
| | siRNA-4 | 2.3 | 32.7 | 0.13 | 11.7 |
| | siRNA-5 | 3.1* | 30.9 | 0.18* | 13.2 |
| | siRNA-6 | 2.9* | 29.7 | 0.22* | 12.5 |
| | siRNA-7 | 2.6* | 29.7 | 0.23* | 11.7 |
| | siRNA-8 | 2.5* | 30.6 | 0.25* | 12.0 |
| NC | m-siRNA-NC | 2.0 | 31.5 | 0.12 | 11.2 |
| group | | | | | |
| VC | BTDS | 2.4 | 35.6 | 0.13 | 11.8 |
| group | | | | | |
| RNAi | m-siRNA-1 | 3.5* | 30.7 | 0.23* | 11.9 |
| group | m-siRNA-2 | 3.3* | 30.2 | 0.17* | 10.3 |
| | m-siRNA-3 | 3.6* | 34.2 | 0.21* | 12.4 |
| | m-siRNA-4 | 2.3 | 32.8 | 0.13 | 10.4 |
| | m-siRNA-5 | 3.5* | 34.1 | 0.20* | 11.2 |
| | m-siRNA-6 | 3.2* | 32.7 | 0.18* | 12.4 |
| | m-siRNA-7 | 2.8* | 29.8 | 0.21* | 12.9 |
| | m-siRNA-8 | 2.7* | 32.8 | 0.23* | 11.8 |
| NC | shRNA (p)-NC | 2.2 | 30.8 | 0.11 | 11.0 |
| group | | | | | |
| VC | BTDS | 2.5 | 32.6 | 0.12 | 11.2 |
| group | | | | | |
| RNAi | shRNA (p)-1 | 3.3* | 31.9 | 0.22* | 12.4 |
| group | shRNA (p)-2 | 2.7* | 31.8 | 0.19* | 13.2 |
| | shRNA (p)-3 | 3.5* | 30.3 | 0.23* | 11.7 |
| | shRNA (p)-4 | 1.9 | 29.8 | 0.13 | 11.6 |
| | shRNA (p)-5 | 3.5* | 31.4 | 0.14* | 12.8 |
| | shRNA (p)-6 | 2.6* | 30.6 | 0.15* | 12.2 |
| | shRNA (p)-7 | 2.5* | 29.5 | 0.14* | 13.2 |
| | shRNA (p)-8 | 2.7* | 30.3 | 0.15* | 12.5 |

Note:
*P < 0.05 represented that compared with the VC or NC group, there was statistically significant difference.

The results of Table 15 showed that the level of bone formation biochemical marker serum PINP was not significantly increased until day 5 after the treatment with the nucleic acid of the invention, the pattern of which was consistent with the time-course changes in mRNA expression levels of BSP and OC after the treatment with the nucleic acid of the invention. On the other hand, the level of bone resorption biochemical marker urine DPD did not change by the treatment with the nucleic acid of the invention. Thus, it suggested that the nucleic acid of the invention could promote bone formation in rats and mice in vivo without stimulating bone resorption.

### Test Embodiment 4

The test embodiment was used for evaluating the anabolic effect of the pharmaceutical composition obtained in the preparation embodiment 4 on healthy rodent bone in vivo.

In this assay, 30 6-month-old female Sprague-Dawley rats or 40 4-month-old female C57/BL mice were divided into nucleic acid treatment groups (m-siRNA-1 group, m-siRNA-3 group and m-siRNA-5 group), non-specific nucleic acid control group (NC group, namely the group injected with m-siRNA-NC) and vehicle control group (VC group), wherein each group contained 6 rats or 8 mice. The pharmaceutical composition obtained in the preparation embodiment 4 was respectively injected into rats or mice in the RNAi group and NC group, and the animals in the VC group were only subject to BTDS injection. The injection dosage of the nucleic acid was 4 mg/kg in rats, and 7.5 mg/kg in mice. All the animals in each group were administrated every week, and six periodic intravenous injections were completed in total. At 6 weeks after the first injection, all the animals were euthanized. Before treatment, another 6 rats or 8 mice were euthanized as baseline group (BS group). Before sacrifice, all the animals were subject to intraperitoneal injection of calcein green (10 mg/kg) and xylenol orange (30 mg/kg) in a time sequence of 10 and 2 days before euthanasia. After sacrifice, the right distal femurs from healthy rats were subjected to micro-CT (viva CT40, SCANCO MEDICAL, Switzerland) analysis, the right distal femurs and the mid-shaft femurs from healthy rats were collected for histomorphometric analysis. In addition, the right distal femurs and the 5^{th} lumbar vertebrae bodies from healthy mice were subjected to micro-CT analysis, the right distal femurs from healthy mice were collected for histomorphometric analysis. The results were as shown in Table 16 (healthy rats) and Table 17 (healthy mice).

TABLE 16

| | | | | m- | m- | m- |
|---|---|---|---|---|---|---|
| | BS | VC | NC | siRNA- | siRNA- | siRNA- |
| Parameters | group | group | group | 1 | 3 | 5 |

Micro-CT measurements at distal femur

| | | | | | | |
|---|---|---|---|---|---|---|
| BMD | 236.09 | 252.36 | 259.77 | 326.03* | 318.07* | 309.32* |
| (mg/cm³) | | | | | | |
| BV/TV | 23.72 | 26.47 | 26.41 | 32.19* | 31.68* | 29.07* |
| (%) | | | | | | |
| Tb · Th | 76.43 | 89.95 | 87.59 | 129.32* | 125.25* | 119.03* |
| (μm) | | | | | | |
| Tb · Sp | 293.93 | 272.68 | 270.64 | 227.29* | 238.08* | 246.87* |
| (μm) | | | | | | |
| Tb · N | 3.58 | 3.67 | 3.51 | 4.09* | 3.84* | 3.79* |
| (l/mm) | | | | | | |
| Conn · D | 51.85 | 63.86 | 63.92 | 72.43* | 66.76* | 65.21* |
| (l/mm³) | | | | | | |
| SMI | 1.77 | 1.58 | 1.55 | 1.09* | 1.11* | 1.09* |

Histomorphometric analysis at distal femur

| | | | | | | |
|---|---|---|---|---|---|---|
| MAR | 1.14 | 1.25 | 1.18 | 1.45* | 1.55* | 1.53* |
| (μm/d) | | | | | | |
| BFR/BS | 0.04 | 0.05 | 0.04 | 0.07* | 0.08* | 0.08* |
| (μm³/ | | | | | | |
| μm²/d) | | | | | | |
| MS/BS | 4.85 | 4.66 | 4.75 | 7.36* | 7.28* | 7.32* |
| (%) | | | | | | |
| Ob · S/BS | 1.92 | 1.76 | 1.94 | 2.82* | 2.76* | 2.78* |
| (%) | | | | | | |
| Oc · S/BS | 1.87 | 1.92 | 1.85 | 2.02 | 2.04 | 2.03 |
| (%) | | | | | | |

Histomorphometric analysis at the mid-shaft femur

| | | | | | | |
|---|---|---|---|---|---|---|
| Ec · BFR/ | 0.76 | 0.82 | 0.85 | 1.21* | 1.23* | 1.25* |
| BS (μm³/ | | | | | | |
| μm²/d) | | | | | | |
| Ps · BFR/ | 0.67 | 0.75 | 0.71 | 1.05* | 1.07* | 1.06* |
| BS (μm³/ | | | | | | |
| μm²/d) | | | | | | |
| Ec · Pm | 7.86 | 8.40 | 8.53 | 7.70 | 7.66 | 7.68 |
| (mm) | | | | | | |
| Ps · Pm | 12.12 | 12.55 | 13.19 | 14.89* | 14.92* | 15.03* |
| (mm) | | | | | | |
| Ct · Th | 0.52 | 0.62 | 0.61 | 0.84* | 0.86* | 0.85* |
| (mm) | | | | | | |
| BA (mm²) | 6.86 | 6.93 | 8.05 | 13.00* | 13.02* | 12.92* |

Note:
*P < 0.05 represented that compared with the VC or NC group, there was statistically significant difference.

TABLE 17

| | | | | m- | m- | m- |
|---|---|---|---|---|---|---|
| | BS | VC | NC | siRNA- | siRNA- | siRNA- |
| Parameter | group | group | group | 1 | 3 | 5 |

Micro-CT measurements at distal femur

| | | | | | | |
|---|---|---|---|---|---|---|
| BMD | 198.04 | 234.53 | 223.4 | 312.5* | 291.04* | 289.56* |
| (mg/cm³) | | | | | | |
| BV/TV | 9.37 | 11.56 | 10.75 | 18.21* | 15.68* | 15.32* |
| (%) | | | | | | |
| Tb · Th | 38.01 | 47.64 | 46.67 | 81.25* | 74.88* | 69.65* |
| (μm) | | | | | | |
| Tb · Sp | 365.21 | 319.71 | 324.77 | 232.19* | 247.19* | 253.83* |
| (μm) | | | | | | |
| Tb · N | 5.89 | 6.41 | 6.22 | 8.21* | 7.80* | 8.02* |
| (l/mm) | | | | | | |
| Conn · D | 269.05 | 306.55 | 311.46 | 367.29* | 355.97* | 349.39* |
| (l/mm³) | | | | | | |
| SMI | 1.57 | 1.76 | 1.85 | 0.96* | 1.16* | 1.32* |

Micro-CT measurements at 5^{th} lumber vertebrae body

| | | | | | | |
|---|---|---|---|---|---|---|
| BMD | 228.02 | 246.27 | 236.25 | 332.28* | 319.04* | 298.31* |
| (mg/cm³) | | | | | | |
| BV/TV | 17.26 | 19.83 | 20.57 | 29.42* | 26.87* | 23.83* |
| (%) | | | | | | |
| Tb · Th | 35.23 | 39.52 | 40.29 | 59.38* | 56.36* | 57.21* |
| (μm) | | | | | | |
| Tb · Sp | 185.57 | 172.87 | 164.8 | 119.39* | 122.16* | 125.82* |
| (μm) | | | | | | |
| Tb · N | 5.27 | 6.04 | 5.76 | 8.21* | 7.35* | 7.19* |
| (l/mm) | | | | | | |
| Conn · D | 206.98 | 216.58 | 221.97 | 298.37* | 289.42* | 290.12* |
| (l/mm³) | | | | | | |
| SMI | 0.74 | 0.76 | 0.69 | 0.55* | 0.52* | 0.61* |

Histomorphometric analysis at distal femur

| | | | | | | |
|---|---|---|---|---|---|---|
| MAR | 0.58 | 0.72 | 0.68 | 0.90* | 0.95* | 0.94* |
| (μm/d) | | | | | | |
| BFR/BS | 0.24 | 0.29 | 0.23 | 0.39* | 0.42* | 0.40* |
| (μm³/ | | | | | | |
| μm²/d) | | | | | | |
| MS/BS | 27.17 | 26.07 | 29.1 | 37.86* | 38.25* | 37.98* |
| (%) | | | | | | |
| Ob · S/BS | 4.80 | 5.53 | 5.42 | 7.64* | 7.79* | 7.70* |
| (%) | | | | | | |
| Oc · S/BS | 3.93 | 4.03 | 3.88 | 4.20 | 4.27 | 4.22 |
| (%) | | | | | | |

Note:
*P < 0.05 represented that compared with the VC or NC group, there was statistically significant difference.

### Test Embodiment 5

The test embodiment was used for evaluating the anabolic effect of the pharmaceutical composition obtained in the preparation embodiment 4 on ovariectomy-induced osteoporotic mouse bone in vivo.

In this assay, 48 4-month-old female C57BL/6J mice were ovariectomized (OVX, n=36) or sham-operated (SHAM, n=12), and were not subject to any treatment within 4 weeks. Before treatment, 6 OVX (OVX-BS) and 6 SHAM (SHAM-BS) mice were euthanized for confirmation of osteoporosis establishment as baseline. Thereafter, the remaining OVX mice were respectively subject to injection with the pharmaceutical composition obtained in the preparation embodiment 4 (OVX-m-siRNA-1 group, OVX-m-siRNA-3 group, OVX-m-siRNA-5 group and OVX-NC group (namely the OVX control group injected with m-siRNA-NC) and injection with BTDS only (OVX-VC group); and the remaining SHAM mice were only subject to injection with the BTDS (SHAM-VC group). The injection dosage of the nucleic acid was 7.5 mg/kg in mice, six animals in each group were administrated every week, and six periodic intravenous injections were completed in total. At 6 weeks after the first injection, all the animals were euthanized. Before sacrifice, all the animals were subject to intraperitoneal injection of calcein green (10 mg/kg) and xylenol orange (30 mg/kg) in a time sequence of 10 and 2 days before euthanasia. After sacrifice, right femurs of the mice were collected for examining trabecular bone at the distal femurs using micro-CT and histomorphologic analysis. The results were as shown in Table 18.

TABLE 18

| | | | | | | | | |
|---|---|---|---|---|---|---|---|---|
| | SHAM- | OVX- | SHAM- | OVX- | OVX- | OVX-m- | OVX-m- | OVX-m- |
| Parameter | BS | BS | VC | VC | NC | siRNA-1 | siRNA-3 | siRNA-5 |

Micro-CT measurements

| | | | | | | | | |
|---|---|---|---|---|---|---|---|---|
| BMD (mg/cm³) | 218.18 | 139.10 | 226.94* | 122.21 | 120.23 | 198.32* | 186.23* | 180.58* |
| BV/TV (%) | 9.42 | 5.78 | 10.66* | 4.87 | 5.23 | 8.23* | 7.87* | 7.96* |
| Tb · Th (μm) | 59.53 | 39.63 | 64.83* | 34.93 | 36.78 | 59.21* | 56.18* | 50.25* |
| Tb · Sp (μm) | 320.18 | 441.63 | 327.48* | 414.83 | 404.50 | 328.32* | 346.53* | 354.9* |
| Tb · N (l/mm) | 6.13 | 3.10 | 6.37* | 3.04 | 3.09 | 4.09* | 4.18* | 3.98* |
| Conn · D | 421.12 | 256.08 | 423.59* | 259.07 | 249.67 | 320.38* | 330.96* | 319.32* |
| (l/mm³) | | | | | | | | |
| SMI | 1.57 | 2.09 | 1.62* | 2.25 | 2.27 | 1.87* | 1.82* | 1.85* |

Histomorphologic analysis

| | | | | | | | | |
|---|---|---|---|---|---|---|---|---|
| MAR (μm/d) | 0.79 | 1.12 | 0.83* | 0.99 | 1.01 | 1.36* | 1.42* | 1.37* |
| BFR/BS | 0.59 | 0.86 | 0.60* | 0.75 | 0.73 | 1.02* | 1.05* | 1.04* |
| (μm³/μm²/d) | | | | | | | | |
| MS/BS (%) | 27.80 | 34.12 | 29.31* | 31.60 | 31.71 | 41.98* | 43.25* | 42.05* |
| Ob · S/BS (%) | 5.99 | 7.01 | 6.03* | 6.60 | 6.45 | 8.68* | 8.82* | 8.76* |
| Oc · S/BS (%) | 5.70 | 8.43 | 5.66 | 7.41 | 7.38 | 7.01 | 7.11 | 7.12 |

Note:
*P < 0.05 represented that compared with the OVX-VC or OVX-NC, there was statistically significant difference.

From the results of the micro-CT and histomorphologic analysis in the test embodiment 4 and the test embodiment 5, it could be analysized that: after injection, the nucleic acid of the invention could significantly increase bone mineral density (BMD), relative bone volume (BV/TV) and trabecular bone parameters (trabecular thickness (Tb.Th), trabecular number (Tb.N), trabecular connectivity density (Conn.D) and the like) in both healthy rodents (including rats and mice) and osteoporotic mice, and also significantly increase osteoblast surfaces/bone surfaces (Ob.S/BS), bone mineralization surfaces/bone surfaces (MS/BS), bone formation rate (BFR/BS) and bone mineral apposition rate (MAR) at the same time. This further indicated that the pharmaceutical composition of the invention could promote the differentiation from bone marrow stromal cells (BMSCs) to osteoblasts and/or recruitment of the osteoblasts on the bone formation surfaces, and also improve the activity of the osteoblasts, thereby promoting the bone formation.

### Test Embodiment 6

The mouse serum obtained in the test embodiment 5 was collected for immunostimulatory analysis. Then the expression levels of the inflammatory cytokine including IFN-α, IFN-γ, TNF-α and IL-6 were dected by using ELISA kits (BD OptEIA™ Mouse TNF ELISA Kit, Catalog No. 560478; BD OptEIA™ Mouse IFN-γ ELISA Kit II, Catalog No. 558258; BD OptEIA™ Mouse IL-6 ELISA Kit, Catalog No. 550950; the above reagents were purchased from BD Bioscience Company; Mouse IFN-α Platinum ELISA, Catalog No. BMS6027, purchased from eBioscience Company), according to the manufacturer's manuals. The results were as shown in Table 19.

TABLE 19

| | SHAM- | OVX- | SHAM- | OVX- | OVX- | OVX-m- | OVX-m- | OVX-m- |
|---|---|---|---|---|---|---|---|---|
| | BS | BS | VC | VC | NC | siRNA-1 | siRNA-3 | siRNA-5 |
| IFN-α (pg/ml) | 0.60 | 0.63 | 1.08 | 1.05 | 1.02 | 1.10 | 1.20 | 1.32 |
| IFN-γ (pg/ml) | 0.20 | 0.30 | 0.30 | 0.37 | 0.35 | 0.29 | 0.34 | 0.35 |
| TNF-α (pg/ml) | 0.90 | 1.03 | 1.38 | 1.35 | 1.32 | 1.20 | 1.40 | 1.30 |
| IL-6 (pg/ml) | 0.20 | 0.24 | 0.30 | 0.30 | 0.27 | 0.25 | 0.28 | 0.29 |

The results of the Table above showed that the pharmaceutical composition of the invention could not cause immunostimulatory activity in vivo.

The results of the test embodiments above indicated that, the nucleic acid provided by the invention showed relatively high inhibition efficiency against CKIP-1 across human, rhesus, rats and mice.

Furthermore, siRNA-1, siRNA-3 and siRNA-5 had higher inhibition efficiency than siRNA-2, siRNA-4, siRNA-6, siRNA-7 and siRNA-8; m-siRNA-1, m-siRNA-3 and m-siRNA-5 had higher inhibition efficiency than m-siRNA-2, m-siRNA-4, m-siRNA-6, m-siRNA-7 and m-siRNA-8; and shRNA (p)-1, shRNA (p)-3 and shRNA (p)-5 had higher inhibition efficiency that shRNA (p)-2, shRNA (p)-4, shRNA (p)-6, shRNA (p)-7 and shRNA (p)-8.

In addition, the modified siRNA (in particular to m-siRNA-1, m-siRNA-3 and m-siRNA-5) had higher inhibition efficiency than the non-modified siRNA (siRNA-1, siRNA-3 and siRNA-5) and shRNA (p) (shRNA (p)-1, shRNA (p)-3 and shRNA (p)-5), indicating that the nucleic acid with specific modification in the preferred embodiment of the invention could exhibit more excellent inhibition effect. Besides, the sequence having the sequence identity of more than 90%, namely in the sense strand, the inconsistent base is positioned at position 19 of the sense strand, and in the antisense strand, the inconsistent base is positioned at position 1 of the antisense strand, e.g. the siRNA (siRNA-1A, siRNA-1G siRNA-1C, siRNA-3A, siRNA-3U, siRNA siRNA-3C, siRNA-5A, siRNA-5U, siRNA-5C) and the modified siRNA (m-siRNA-1A, m-siRNA-1G m-siRNA-1C, m-siRNA-3A, m-siRNA-3U, m-siRNA-3C, m-siRNA-5A, m-siRNA-5U and m-siRNA-5C), had equivalent silencing activities compared to the siRNA having the sequence identity of 100%, e.g. the siRNA (siRNA-1, siRNA-3, siRNA-5) and the modified siRNA (m-siRNA-1, m-siRNA-3 and m-siRNA-5). The result of shRNA was also similar.

The preferred embodiments of the invention are described in detail above. However, the invention is not limited to the specific details in the embodiments, and in the scope of technical concept, the technical proposal of the invention can be subjected to a variety of simple modifications, and these simple modifications still belong to the scope of protection of the invention.

In addition, it needs to be noted that the various specific technical features described in the above embodiments can be combined in any suitable way without contradiction. In order to avoid the unnecessary repetition, the various possible combination ways will not be described any more herein.

In addition, the various different embodiments of the invention can also be combined arbitrarily, and the combinations should also be considered as the contents disclosed in the invention as long as they do not depart from the idea of the invention.

1. A nucleic acid, containing at least one of siRNA-1 with a sense strand sequence which is a sequence having sequence identity of more than 90% with SEQ ID NO: 1 and an antisense strand sequence which is the sequence having the sequence identity of more than 90% with SEQ ID NO: 2, siRNA-2 with the sense strand sequence which is the sequence having the sequence identity of more than 90% with SEQ ID NO: 3 and the antisense strand sequence which is the sequence having the sequence identity of more than 90% with SEQ ID NO: 4, siRNA-3 with the sense strand sequence which is the sequence having the sequence identity of more than 90% with SEQ ID NO: 5 and the antisense strand sequence which is the sequence having the sequence identity of more than 90% with SEQ ID NO: 6, siRNA-4 with the sense strand sequence which is the sequence having the sequence identity of more than 90% with SEQ ID NO: 7 and the antisense strand sequence which is the sequence having the sequence identity of more than 90% with SEQ ID NO: 8, siRNA-5 with the sense strand sequence which is the sequence having the sequence identity of more than 90% with SEQ ID NO: 9 and the antisense strand sequence which is the sequence having the sequence identity of more than 90% with SEQ ID NO: 10, siRNA-6 with the sense strand sequence which is the sequence having the sequence identity of more than 90% with SEQ ID NO: 11 and the antisense strand sequence which is the sequence having the sequence identity of more than 90% with SEQ ID NO: 12, siRNA-7 with the sense strand sequence which is the sequence having the sequence identity of more than 90% with SEQ ID NO: 13 and the antisense strand sequence which is the sequence having the sequence identity of more than 90% with SEQ ID NO: 14 and siRNA-8 with the sense strand sequence which is the sequence having the sequence identity of more than 90% with SEQ ID NO: 15 and the antisense strand sequence which is the sequence having the sequence identity of more than 90% with SEQ ID NO: 16.

2. The nucleic acid according to claim 1, wherein the sequence identity of more than 90% means that one base inconsistency exists between the sequences, in the sense strand, one inconsistent base is positioned at position 19 of the sense strand, and in the antisense strand, one inconsistent base is positioned at position 1 of the antisense strand.

3. The nucleic acid according to claim 1, wherein the nucleic acid contains at least one of siRNA-1 with the sense strand sequence of SEQ ID NO: 1 and the antisense strand sequence of SEQ ID NO: 2, siRNA-2 with the sense strand sequence of SEQ ID NO: 3 and the antisense strand sequence of SEQ ID NO: 4, siRNA-3 with the sense strand sequence of SEQ ID NO: 5 and the antisense strand sequence of SEQ ID NO: 6, siRNA-4 with the sense strand sequence of SEQ ID NO: 7 and the antisense strand sequence of SEQ ID NO: 8, siRNA-5 with the sense strand sequence of SEQ ID NO: 9 and the antisense strand sequence of SEQ ID NO: 10, siRNA-6 with the sense strand sequence of SEQ ID NO: 11 and the antisense strand sequence of SEQ ID NO: 12, siRNA-7 with the sense strand sequence of SEQ ID NO: 13 and the antisense strand sequence of SEQ ID NO: 14, siRNA-8 with the sense strand sequence of SEQ ID NO: 15 and the antisense strand sequence of SEQ ID NO: 16, siRNA-1A with the sense strand sequence of SEQ ID NO: 83 and the antisense strand sequence of SEQ ID NO: 84, siRNA-1G with the sense strand sequence of SEQ ID NO: 85 and the antisense strand sequence of SEQ ID NO: 86, siRNA-1C with the sense strand sequence of SEQ ID NO: 87 and the antisense strand sequence of SEQ ID NO: 88, siRNA-3A with the sense strand sequence of SEQ ID NO: 89 and the antisense strand sequence of SEQ ID NO: 90, siRNA-3U with the sense strand sequence of SEQ ID NO: 91 and the antisense strand sequence of SEQ ID NO: 92, siRNA-3C with the sense strand sequence of SEQ ID NO: 93 and the antisense strand sequence of SEQ ID NO: 94, siRNA-5A with the sense strand sequence of SEQ ID NO: 95 and the antisense strand sequence of SEQ ID NO: 96, siRNA-5U with the sense strand sequence of SEQ ID NO: 97 and the antisense strand sequence of SEQ ID NO: 98 and siRNA-5C with the sense strand sequence of SEQ ID NO: 99 and the antisense strand sequence of SEQ ID NO: 100.

4. The nucleic acid according to claim 1, wherein the nucleic acid contains at least one modified nucleotide group, and the modified nucleotide group is the nucleotide group with a modified phosphoric acid group and/or a ribose group.

5. The nucleic acid according to claim 4, wherein the nucleotide group with the modified ribose group is the nucleotide group with the ribose group of which 2′-OH is substituted by methoxy or fluoro group.

6. The nucleic acid according to claim 5, wherein the nucleotide group containing a uracil base or a cytosine base in the sense strand of the nucleic acid is the nucleotide group with the modified ribose group, and 3′ ends of the sense strand and the antisense strand of the nucleic acid are connected with dTdT, respectively.

7-9. (canceled)

10. A pharmaceutical composition, containing the nucleic acid of claim 1 and a pharmaceutically acceptable carrier.

11. The pharmaceutical composition according to claim 10, wherein the pharmaceutically acceptable carrier is the vector covalently linking a liposome and bone-targeted molecules, and the molar ratio of the part of the bone-targeted molecules to the part of the liposome is (2-10): 100.

12. The pharmaceutical composition according to claim 11, wherein the molar ratio of the nucleic acid to the part of the liposome is (5-10): 1, wherein the molar amount of the nucleic acid is calculated by element P, and the molar amount of the part of the liposome is calculated by element N.

13. The pharmaceutical composition according to claim 11, wherein the liposome contains 1, 2-dioleoyl-3-trimethylammonium-propane, dioleoyl phosphatidylethanolamine, cholesterol, distearoyl phosphoethanolamine-methoxypolyethylene glycol 2000 and distearoyl phosphoethanolamine-polyethylene glycol 2000-maleimide, and the molar ratio of the substances is (20-25):(6-8):(15-20):(1-2):1.

14. The pharmaceutical composition according to claim 11, wherein the bone-targeted molecules are a polypeptide with an amino acid sequence as shown in SEQ ID NO: 82.

15-16. (canceled)

17. A method for treating and/or preventing diseases related to the abnormal expression of CKIP-1 gene, the method comprising performing administration on a patient by using the nucleic acid of claim 1.

18. The method according to claim 17, wherein the diseases related to the abnormal expression of CKIP-1 gene include at least one of osteoporosis, osteoporotic fracture, fracture healing retardation, bone necrosis, degenerative arthritis and rheumatoid arthritis late-stage bone destruction.

19-20. (canceled)

21. A method for treating and/or preventing diseases related to the abnormal expression of CKIP-1 gene, the method comprising performing administration on a patient by using the pharmaceutical composition of claim 10.


SEQUENCE LISTING (118 sequences)

SEQ ID NO: 1 - DNA, 21 bp, Artificial
  The sequence is synthesized
ccucuugugcugagagcuutt

SEQ ID NO: 2 - DNA, 21 bp, Artificial
  The sequence is synthesized
aagcucucagcacaagaggtt

SEQ ID NO: 3 - DNA, 21 bp, Artificial
  The sequence is synthesized
ugagagaccuguacagacatt

SEQ ID NO: 4 - DNA, 21 bp, Artificial
  The sequence is synthesized
ugucuguacaggucucucatt

SEQ ID NO: 5 - DNA, 21 bp, Artificial
  The sequence is synthesized
ccugagugacuaugagaagtt

SEQ ID NO: 6 - DNA, 21 bp, Artificial
  The sequence is synthesized
cuucucauagucacucaggtt

SEQ ID NO: 7 - DNA, 21 bp, Artificial
  The sequence is synthesized
ccggaaauucugcgggaaatt

SEQ ID NO: 8 - DNA, 21 bp, Artificial
  The sequence is synthesized
uuucccgcagaauuuccggtt

SEQ ID NO: 9 - DNA, 21 bp, Artificial
  The sequence is synthesized
ggaugaggucaccguugagtt

SEQ ID NO: 10 - DNA, 21 bp, Artificial
  The sequence is synthesized
cucaacggugaccucaucctt

SEQ ID NO: 11 - DNA, 21 bp, Artificial
  The sequence is synthesized
gugcugagagcuuucgggutt

SEQ ID NO: 12 - DNA, 21 bp, Artificial
  The sequence is synthesized
acccgaaagcucucagcactt

SEQ ID NO: 13 - DNA, 21 bp, Artificial
  The sequence is synthesized
ggucggcuggguccggaaatt

SEQ ID NO: 14 - DNA, 21 bp, Artificial
  The sequence is synthesized
uuuccggacccagccgacctt

SEQ ID NO: 15 - DNA, 21 bp, Artificial
  The sequence is synthesized
accgcuauguggugcugaatt

SEQ ID NO: 16 - DNA, 21 bp, Artificial
  The sequence is synthesized
uucagcaccacauagcggutt

SEQ ID NO: 17 - DNA, 19 bp, Artificial
  The sequence is synthesized
cctcttgtgctgagagctt

SEQ ID NO: 18 - DNA, 19 bp, Artificial
  The sequence is synthesized
aagctctcagcacaagagg

SEQ ID NO: 19 - DNA, 19 bp, Artificial
  The sequence is synthesized
tgagagacctgtacagaca

SEQ ID NO: 20 - DNA, 19 bp, Artificial
  The sequence is synthesized
tgtctgtacaggtctctca

SEQ ID NO: 21 - DNA, 19 bp, Artificial
  The sequence is synthesized
cctgagtgactatgagaag

SEQ ID NO: 22 - DNA, 19 bp, Artificial
  The sequence is synthesized
cttctcatagtcactcagg

SEQ ID NO: 23 - DNA, 19 bp, Artificial
  The sequence is synthesized
ccggaaattctgcgggaaa

SEQ ID NO: 24 - DNA, 19 bp, Artificial
  The sequence is synthesized
tttcccgcagaatttccgg

SEQ ID NO: 25 - DNA, 19 bp, Artificial
  The sequence is synthesized
ggatgaggtcaccgttgag

SEQ ID NO: 26 - DNA, 19 bp, Artificial
  The sequence is synthesized
ctcaacggtgacctcatcc

SEQ ID NO: 27 - DNA, 19 bp, Artificial
  The sequence is synthesized
gtgctgagagctttcgggt

SEQ ID NO: 28 - DNA, 19 bp, Artificial
  The sequence is synthesized
acccgaaagctctcagcac

SEQ ID NO: 29 - DNA, 19 bp, Artificial
  The sequence is synthesized
ggtcggctgggtccggaaa

SEQ ID NO: 30 - DNA, 19 bp, Artificial
  The sequence is synthesized
tttccggacccagccgacc

SEQ ID NO: 31 - DNA, 19 bp, Artificial
  The sequence is synthesized
accgctatgtggtgctgaa

SEQ ID NO: 32 - DNA, 19 bp, Artificial
  The sequence is synthesized
ttcagcaccacatagcggt

SEQ ID NO: 33 - RNA, 19 nt, Artificial
  The sequence is synthesized
ccucuugugcugagagcuu

SEQ ID NO: 34 - RNA, 19 nt, Artificial
  The sequence is synthesized
ugagagaccuguacagaca

SEQ ID NO: 35 - RNA, 19 nt, Artificial
  The sequence is synthesized
ccugagugacuaugagaag

SEQ ID NO: 36 - RNA, 19 nt, Artificial
  The sequence is synthesized
ccggaaauucugcgggaaa

SEQ ID NO: 37 - RNA, 19 nt, Artificial
  The sequence is synthesized
ggaugaggucaccguugag

SEQ ID NO: 38 - RNA, 19 nt, Artificial
  The sequence is synthesized
gugcugagagcuuucgggu

SEQ ID NO: 39 - RNA, 19 nt, Artificial
  The sequence is synthesized
ggucggcuggguccggaaa

SEQ ID NO: 40 - RNA, 19 nt, Artificial
  The sequence is synthesized
accgcuauguggugcugaa

SEQ ID NO: 41 - DNA, 21 bp, Artificial
  The sequence is synthesized
uucuccgaacgugucacgutt

SEQ ID NO: 42 - DNA, 21 bp, Artificial
  The sequence is synthesized
acgugacacguucggagaatt

SEQ ID NO: 43 - DNA, 19 bp, Artificial
  The sequence is synthesized
ttctccgaacgtgtcacgt

SEQ ID NO: 44 - DNA, 19 bp, Artificial
  The sequence is synthesized
acgtgacacgttcggagaa

SEQ ID NO: 45 - DNA, 20 bp, Artificial
  The sequence is synthesized
acccgagccaagaaccgtat

SEQ ID NO: 46 - DNA, 20 bp, Artificial
  The sequence is synthesized
tggaagccacagccattagg

SEQ ID NO: 47 - DNA, 21 bp, Artificial
  The sequence is synthesized
ggcatggactgtggtcatgag

SEQ ID NO: 48 - DNA, 20 bp, Artificial
  The sequence is synthesized
tgcaccaccaactgcttagc

SEQ ID NO: 49 - DNA, 18 bp, Artificial
  The sequence is synthesized
tcacccgagccaagaacc

SEQ ID NO: 50 - DNA, 19 bp, Artificial
  The sequence is synthesized
ggaagccacagccattagg

SEQ ID NO: 51 - DNA, 18 bp, Artificial
  The sequence is synthesized
tgacctgccgtctggaaa

SEQ ID NO: 52 - DNA, 18 bp, Artificial
  The sequence is synthesized
gggtgtcgctgttgaagt

SEQ ID NO: 53 - DNA, 20 bp, Artificial
  The sequence is synthesized
gagctttcgggtcgatctgg

SEQ ID NO: 54 - DNA, 20 bp, Artificial
  The sequence is synthesized
ggctcccttgtctggtcttt

SEQ ID NO: 55 - DNA, 20 bp, Artificial
  The sequence is synthesized
caagttcaacggcacagtca

SEQ ID NO: 56 - DNA, 20 bp, Artificial
  The sequence is synthesized
ccatttgatgttagcgggat

SEQ ID NO: 57 - DNA, 20 bp, Artificial
  The sequence is synthesized
aaccgctatgtggtgctgaa

SEQ ID NO: 58 - DNA, 21 bp, Artificial
  The sequence is synthesized
cagggtgaacttgctgtgatt

SEQ ID NO: 59 - DNA, 19 bp, Artificial
  The sequence is synthesized
tgcaccaccaactgcttag

SEQ ID NO: 60 - DNA, 19 bp, Artificial
  The sequence is synthesized
ggatgcagggatgatgttc

SEQ ID NO: 61 - DNA, 20 bp, Artificial
  The sequence is synthesized
tggacggtgaacgggagaac

SEQ ID NO: 62 - DNA, 20 bp, Artificial
  The sequence is synthesized
ggacgccgtgaagcaggtga

SEQ ID NO: 63 - DNA, 20 bp, Artificial
  The sequence is synthesized
cctacagcacgcttgtggat

SEQ ID NO: 64 - DNA, 20 bp, Artificial
  The sequence is synthesized
attgggatggagggagttta

SEQ ID NO: 65 - DNA, 25 bp, Artificial
  The sequence is synthesized
agaaacggatgactttaagcaagaa

SEQ ID NO: 66 - DNA, 21 bp, Artificial
  The sequence is synthesized
tctctgcatggtctccatcgt

SEQ ID NO: 67 - DNA, 20 bp, Artificial
  The sequence is synthesized
agaaagagcagcacggttga

SEQ ID NO: 68 - DNA, 20 bp, Artificial
  The sequence is synthesized
ccctcgtagccttcatagcc

SEQ ID NO: 69 - DNA, 20 bp, Artificial
  The sequence is synthesized
ctcactctgctggccctgac

SEQ ID NO: 70 - DNA, 20 bp, Artificial
  The sequence is synthesized
ccttactgccctcctgcttg

SEQ ID NO: 71 - DNA, 21 bp, Artificial
  The sequence is synthesized
atctttggtctggctcccatg

SEQ ID NO: 72 - DNA, 19 bp, Artificial
  The sequence is synthesized
tttcccgttcaccgtccac

SEQ ID NO: 73 - DNA, 18 bp, Artificial
  The sequence is synthesized
cctggtaaagatggtgcc

SEQ ID NO: 74 - DNA, 22 bp, Artificial
  The sequence is synthesized
caccaggttcacctttcgcacc

SEQ ID NO: 75 - DNA, 21 bp, Artificial
  The sequence is synthesized
acactttcactccaatcgtcc

SEQ ID NO: 76 - DNA, 20 bp, Artificial
  The sequence is synthesized
tgccctttccgttgttgtcc

SEQ ID NO: 77 - DNA, 21 bp, Artificial
  The sequence is synthesized
aagcagcaccgttgagtatgg

SEQ ID NO: 78 - DNA, 25 bp, Artificial
  The sequence is synthesized
ccttgtagtagctgtattcgtcctc

SEQ ID NO: 79 - DNA, 24 bp, Artificial
  The sequence is synthesized
gcaataaggtagtgaacagactcc

SEQ ID NO: 80 - DNA, 21 bp, Artificial
  The sequence is synthesized
gtttgtaggcggtcttcaagc

SEQ ID NO: 81 - DNA, 8 bp, Artificial
  The sequence is synthesized
tcaagaga

SEQ ID NO: 82 - protein, 18 aa, Artificial
  The sequence is synthesized
DSSDSSDSSDSSDSSDSS

SEQ ID NO: 83 - DNA, 21 bp, Artificial
  The sequence is synthesized
ccucuugugcugagagcuatt

SEQ ID NO: 84 - DNA, 21 bp, Artificial
  The sequence is synthesized
uagcucucagcacaagaggtt

SEQ ID NO: 85 - DNA, 21 bp, Artificial
  The sequence is synthesized
ccucuugugcugagagcugtt

SEQ ID NO: 86 - DNA, 21 bp, Artificial
  The sequence is synthesized
cagcucucagcacaagaggtt

SEQ ID NO: 87 - DNA, 21 bp, Artificial
  The sequence is synthesized
ccucuugugcugagagcuctt

SEQ ID NO: 88 - DNA, 21 bp, Artificial
  The sequence is synthesized
gagcucucagcacaagaggtt

SEQ ID NO: 89 - DNA, 21 bp, Artificial
  The sequence is synthesized
ccugagugacuaugagaaatt

SEQ ID NO: 90 - DNA, 21 bp, Artificial
  The sequence is synthesized
uuucucauagucacucaggtt

SEQ ID NO: 91 - DNA, 21 bp, Artificial
  The sequence is synthesized
ccugagugacuaugagaautt

SEQ ID NO: 92 - DNA, 21 bp, Artificial
  The sequence is synthesized
auucucauagucacucaggtt

SEQ ID NO: 93 - DNA, 21 bp, Artificial
  The sequence is synthesized
ccugagugacuaugagaactt

SEQ ID NO: 94 - DNA, 21 bp, Artificial
  The sequence is synthesized
guucucauagucacucaggtt

SEQ ID NO: 95 - DNA, 21 bp, Artificial
  The sequence is synthesized
ggaugaggucaccguugaatt

SEQ ID NO: 96 - DNA, 21 bp, Artificial
  The sequence is synthesized
uucaacggugaccucaucctt

SEQ ID NO: 97 - DNA, 21 bp, Artificial
  The sequence is synthesized
ggaugaggucaccguugautt

SEQ ID NO: 98 - DNA, 21 bp, Artificial
  The sequence is synthesized
aucaacggugaccucaucctt

SEQ ID NO: 99 - DNA, 21 bp, Artificial
  The sequence is synthesized
ggaugaggucaccguugactt

SEQ ID NO: 100 - DNA, 21 bp, Artificial
  The sequence is synthesized
gucaacggugaccucaucctt

SEQ ID NO: 101 - DNA, 19 bp, Artificial
  The sequence is synthesized
cctcttgtgctgagagcta

SEQ ID NO: 102 - DNA, 19 bp, Artificial
  The sequence is synthesized
tagctctcagcacaagagg

SEQ ID NO: 103 - DNA, 19 bp, Artificial
  The sequence is synthesized
cctcttgtgctgagagctg

SEQ ID NO: 104 - DNA, 19 bp, Artificial
  The sequence is synthesized
cagctctcagcacaagagg

SEQ ID NO: 105 - DNA, 19 bp, Artificial
  The sequence is synthesized
cctcttgtgctgagagctc

SEQ ID NO: 106 - DNA, 19 bp, Artificial
  The sequence is synthesized
gagctctcagcacaagagg

SEQ ID NO: 107 - DNA, 19 bp, Artificial
  The sequence is synthesized
cctgagtgactatgagaaa

SEQ ID NO: 108 - DNA, 19 bp, Artificial
  The sequence is synthesized
tttctcatagtcactcagg

SEQ ID NO: 109 - DNA, 19 bp, Artificial
  The sequence is synthesized
cctgagtgactatgagaat

SEQ ID NO: 110 - DNA, 19 bp, Artificial
  The sequence is synthesized
attctcatagtcactcagg

SEQ ID NO: 111 - DNA, 19 bp, Artificial
  The sequence is synthesized
cctgagtgactatgagaac

SEQ ID NO: 112 - DNA, 19 bp, Artificial
  The sequence is synthesized
gttctcatagtcactcagg

SEQ ID NO: 113 - DNA, 19 bp, Artificial
  The sequence is synthesized
ggatgaggtcaccgttgaa

SEQ ID NO: 114 - DNA, 19 bp, Artificial
  The sequence is synthesized
ttcaacggtgacctcatcc

SEQ ID NO: 115 - DNA, 19 bp, Artificial
  The sequence is synthesized
ggatgaggtcaccgttgat

SEQ ID NO: 116 - DNA, 19 bp, Artificial
  The sequence is synthesized
atcaacggtgacctcatcc

SEQ ID NO: 117 - DNA, 19 bp, Artificial
  The sequence is synthesized
ggatgaggtcaccgttgac

SEQ ID NO: 118 - DNA, 19 bp, Artificial
  The sequence is synthesized
gtcaacggtgacctcatcc